(19) United States
(12) Patent Application Publication
(10) Pub. No.: US 2003/0228616 A1
(43) Pub. Date: Dec. 11, 2003

(54) DNA polymerase mutants with reverse transcriptase activity

(75) Inventors: Bahram Arezi, Carlsbad, CA (US); Holly Hogrefe, San Diego, CA (US); Joseph A. Sorge, Wilson, WY (US); Connie Jo Hansen, San Diego, CA (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/435,766

(22) Filed: May 12, 2003

Related U.S. Application Data
(63) Continuation-in-part of application No. 10/223,650, filed on Aug. 19, 2002.
Application No. 10/223,650 is a continuation-in-part of application No. 09/896,923, filed on Jun. 29, 2001.
Application No. 09/896,923 is a continuation-in-part of application No. 09/698,341, filed on Oct. 27, 2000.
(60) Provisional application No. 60/162,600, filed on Oct. 29, 1999.

(30) Foreign Application Priority Data
Oct. 27, 2000 (WO) PCT/US00/29706

(51) Int. Cl. C12Q 1/68; C07H 21/04; C12P 19/34; C12N 9/22; C12N 1/20; C12N 15/74

(52) U.S. Cl. 435/6; 435/69.1; 435/199; 435/252.3; 435/320.1; 435/91.2; 536/23.2

(57) ABSTRACT

The present invention relates to compositions and kits comprising a mutant DNA polymerase with increased reverse transcriptase activity. The invention also relates to methods for using the subject compositions and kits.

### RELATED APPLICATIONS

This application is a Continuation-in-Part of U.S. application Ser. No. 10/223,650, filed Aug. 19, 2002, which is a Continuation-in-Part of U.S. application Ser. No. 09/896,923, filed Jun. 29, 2001, which is a Continuation-in-Part of U.S. Utility Application No. 09/698,341, filed Oct. 27, 2000, which claims the priority of U.S. Provisional Application No. 60/162,600, filed Oct. 29, 1999. This application also claims the priority of International Application No. PCT/U.S. 00/29706, filed Oct. 27, 2000. Each of these applications is incorporated herein by reference in their entirety, including figures and drawings.

### FIELD OF THE INVENTION

The present invention relates to enzymes with reverse transcriptase and DNA polymerase activity.

### BACKGROUND

Reverse transcription (RT) and the polymerase chain reaction (PCR) are critical to many molecular biology and related applications, particularly to gene expression analysis applications. In these applications, reverse transcription is used to prepare template DNA (e.g., cDNA) from an initial RNA sample (e.g. mRNA), which template DNA is then amplified using PCR to produce a sufficient amount of amplified product for the application of interest.

The RT and PCR steps of DNA amplification can be carried out as a two-step or one-step process.

In one type of two-step process, the first step involves synthesis of first strand cDNA with a reverse transcriptase, following by a second PCR step. In certain protocols, these steps are carried out in separate reaction tubes. In these two tube protocols, following reverse transcription of the initial RNA template in the first tube, an aliquot of the resultant product is then placed into the second PCR tube and subjected to PCR amplification.

In a second type of two-step process, both RT and PCR are carried out in the same tube using a compatible RT and PCR buffer. Typically, reverse transcription is carried out first, followed by addition of PCR reagents to the reaction tube and subsequent PCR.

A variety of one-step RT-PCR protocols have been developed, see Blain & Goff, J. Biol. Chem. (1993) 5: 23585-23592; Blain & Goff J. Virol. (1995) 69:4440-4452; Sellner et al., J. Virol. Method. (1994) 49:47-58; PCR, Essential Techniques (ed. J. F. Burke, J. Wiley & Sons, New York)(1996) pp61-63; 80-81.

Some one-step systems are commercially available, for example, SuperScript One-Step RT-PCR System description on the world-wide web at lifetech.com/world_whatsnew/archive/nz₁₋₋₃.html; Access RT-PCR System and Access RT-PCR Introductory System described on the world wide web at promega.com/tbs/tb220/tb220.html; AdvanTaq & AdvanTaq Plus PCR kits and User Manual available at www.clontech.com, and ProSTAR™ HF single-tube RT-PCR kit (Stratagene, Catalog No. 600164, information available on the world wide web at stratagene.com).

Reverse transcription is commonly performed with viral reverse transcriptases isolated from Avian mycloblastosis virus (AMV-RT) or Moloney murine leukemia virus (MMLV-RT), which are active in the presence of magnesium ions.

Certain RT-PCR methods use an enzyme blend or enzymes with both reverse transcriptase and DNA polymerase or exonuclease activities, e.g., as described in U.S. Pat. Nos. 6,468,775; 6,399,320; 5,310,652; 6,300,073; patent application No. U.S. 2002/0119465A1; EP 1,132,470A1 and WO 00/71739A1, all of which are incorporated herein by reference.

Some existing RT-PCR one-step methods utilize the native reverse transcriptase activity of DNA polymerases of thermophilic organisms which are active at higher temperatures, for example, as described in the references cited above herein, and in U.S. Pat. Nos. 5,310,652, 6,399,320, 5,322,770, and 6,436677; Myers and Gelfand, 1991, Biochem., 30:7661-7666; all of which are incorporated herein by reference. Thermostable DNA polymerases with reverse transcriptase activities are commonly isolated from Thermus species.

Recently, U.S. patent application 2002/0012970 (incorporated herein by reference) describes modifying a thermostable DNA polymerase to obtain RT activity for combined RT-PCR reaction.

### SUMMARY OF THE INVENTION

The invention relates to the discovery of thermostable DNA polymerases, e.g., Archacal DNA polymerases, that bear one or more mutations resulting in increased reverse transcriptase activity relative to their unmodified wild-type forms.

In a first aspect, a recombinant mutant Archaeal DNA polymerase is disclosed that exhibits an increased reverse transcriptase activity.

In one embodiment, the Archaeal DNA polymerase is a mutant of an Archaeal DNA polymerase selected from the group of wild-type enzymes consisting of: Thermococcus litoralis DNA polymerase (Vent; SEQ ID NO: 7); Pyrococcus sp. DNA polymerase (Deep Vent; SEQ ID NO: 9); Pyrococcus furiosus DNA polymerase (Pfu; SEQ ID NO: 3); JDF-3 DNA polymerase (SEQ ID NO: 1); Sulfolobus solfataricus DNA polymerase (Sso; GenBank Accession No. NP342079); Thermococcus gorgonarius DNA polymerase (Tgo; SEQ ID NO: 11); Thermococcus species TY DNA polymerase (SEQ ID NO: 13); Thermococcus species strain KOD1 (KOD) DNA polymerase (SEQ ID NO: 5); Sulfolobus acidocaldarius DNA polymerase (GenBank Accession No. P95690); Thermococcus species 9° N-7 DNA polymerase (SEQ ID NO: 15); Pyrodictium occultum DNA polymerase (GenBank Accession No. BAA07580); Methanococcus voltae DNA polymerase (GenBank Accession No. P52025); Methanobacterium thermoautotrophicum DNA polymerase (GenBank Accession No. NP276336); Methanococcus jannaschii DNA polymerase (GenBank Accession No. Q58295); Thermoplasma acidophilum DNA polymerase (GenBank Accession No. NP393515); Pyrobaculum islandicum DNA polymerase (GenBank Accession No. AAF27815); Desulfurococcus strain TOK DNA polymerase (D. Tok Pol; GenBank Accession No. ID5AA); Pyrococcus abyssi DNA polymerase (GenBank Accession No. NP127396); Pyrococcus horikoshii DNA polymerase (GenBank Accession No. 059610); Thermococcus fumicolans DNA polymerase (GenBank Accession No. P74918); and Aeropyrum pernix DNA polymerase (GenBank Accession No. NP 148473).

In a second aspect, a recombinant mutant Archaeal DNA polymerase is disclosed that exhibits an increased reverse transcriptase activity, wherein the wild-type form comprises an amino acid sequence selected from SEQ ID Nos. 1, 3, 5, 7, 9, 11, 13, and 15.

In one embodiment of either of the first or second aspects above, the Archaeal DNA polymerase comprises an amino acid mutation at the amino acid corresponding to L408 of SEQ ID NO: 1.

In another embodiment, the amino acid mutation at the position corresponding to L408 of SEQ ID NO: 1 is a leucine to phenylalanine mutation, leucine to tyrosine mutation, leucine to histidine mutation or a leucine to tryptophan mutation.

In another embodiment, the mutant Archaeal DNA polymerase further exhibits a decreased 3′-5′ exonuclease activity.

In another embodiment, the mutant Archaeal DNA polymerase further exhibits a reduction in non-conventional nucleotide discrimination.

In another aspect, a chimeric polypeptide is disclosed that comprises a mutant Archaeal DNA polymerase and a second polypeptide fused to the mutant Archaeal DNA polymerase, wherein the mutant Archaeal DNA polymerase exhibits an increased reverse transcriptase activity.

In one embodiment, the second polypeptide is fused to the N- or C-terminus of the mutant Archaeal DNA polymerase.

In another embodiment, the second polypeptide is a polynucleotide binding protein.

In another embodiment, the polynucleotide binding protein is selected from the group consisting of: nucleocapsid protein Ncp7, recA, SSB, T4 gene 32 protein, an Archaeal non-sequence specific double stranded DNA binding protein, and a helix-hairpin-helix domain.

In another embodiment, the Archaeal sequence non-specific double stranded DNA binding protein is Sso7d.

In another embodiment, the helix-hairpin-helix domain is from topoisomerase V.

In another aspect, an isolated polynucleotide encoding a mutant Archaeal DNA polymerase is disclosed which exhibits an increased reverse transcriptase activity.

In one embodiment, the Archaeal DNA polymerase is selected from the group of wild-type enzymes consisting of consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9° N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

In another aspect, an isolated polynucleotide encoding a mutant Archaeal DNA polymerase is disclosed which exhibits an increased reverse transcriptase activity compared to a DNA polymerase encoded by a wild-type polynucleotide, wherein the wild-type polynucleotide comprises a sequence selected from the group consisting of SEQ ID Nos. 2, 4, 6, 8, 10, 12, 14, and 16.

In one embodiment of either of the two preceeding aspects, the Archaeal DNA polymerase comprises an amino acid mutation at the amino acid corresponding to L408 of SEQ ID NO: 1.

In another embodiment, the amino acid mutation at the amino acid corresponding to L408 of SEQ ID NO: 1 is a leucine to phenylalanine mutation, leucine to tyrosine mutation, leucine to histidine mutation or a leucine to tryptophan mutation.

In another aspect, an isolated polynucleotide is disclosed that encodes a chimeric polypeptide as described in the preceding aspects.

In another aspect, a composition is disclosed comprising a mutant Archaeal DNA polymerase exhibiting an increased reverse transcriptase activity.

In one embodiment, the Archaeal DNA polymerase is selected from the group of wild-type enzymes consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9° N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

In another aspect, a composition comprising a mutant Archaeal DNA polymerase exhibiting an increased reverse transcriptase activity is disclosed, wherein the wild-type form comprises an amino acid sequence selected from SEQ ID Nos. 1, 3, 5, 7, 9, 11, 13, and 15.

In one embodiment of either of the two preceeding aspects, the Archaeal DNA polymerase comprises an amino acid mutation at the amino acid corresponding to L408 of SEQ ID NO: 1.

In another embodiment, the amino acid mutation at the amino acid corresponding to L408 of SEQ ID NO: 1 is a leucine to phenylalanine mutation, leucine to tyrosine mutation, leucine to histidine mutation or a leucine to tryptophan mutation.

In another embodiment, the composition further comprises one or more reagents selected from the group consisting of: reaction buffer, dNTP, control RNA template and control primers.

In another embodiment, the composition further comprises one or more reagents selected from the group consisting of: formamide, DMSO, betaine, trehalose, low molecular weight amides, sulfones, an Archaeal accessory factor, a single stranded DNA binding protein, a DNA polymerase other than the mutant Archaeal DNA polymerase, another reverse transcriptase enzyme, and an exonuclease.

In another aspect, a kit is disclosed comprising a mutant Archaeal DNA polymerase exhibiting an increased reverse transcriptase activity, and packaging materials therefor.

In one embodiment, the Archaeal DNA polymerase is selected from the group of wild-type enzymes consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9° N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

In another aspect, a kit is disclosed comprising a mutant Archaeal DNA polymerase exhibiting an increased reverse transcriptase activity, wherein the wild-type form comprises an amino acid sequence selected from SEQ ID Nos. 1, 3, 5, 7, 9, 11, 13, and 15.

In one embodiment, the Archaeal DNA polymerase is mutated to comprise an amino acid mutation at the position corresponding to L408 of SEQ ID NO: 1.

In another embodiment, the amino acid mutation at the amino acid corresponding to L408 of SEQ ID NO: 1 is a leucine to phenylalanine mutation, leucine to tyrosine mutation, leucine to histidine mutation or a leucine to tryptophan mutation.

In another embodiment, the kit further comprises one or more reagents selected from the group consisting of: reaction buffer, dNTP, control RNA template and a control primer.

In another embodiment, the kit further comprises one or more reagents selected from the group consisting of: formamide, DMSO, betaine, trehalose, low molecular weight amides, sulfones, an Archaeal accessory factor, a single-stranded DNA binding protein, a DNA polymerase other than the mutant Archaeal DNA polymerase, another reverse transcriptase enzyme, and an exonuclease.

In another aspect, a method for reverse transcribing an RNA template is disclosed, comprising incubating the RNA template in a reaction mixture comprising a mutant Archaeal DNA polymerase exhibiting an increased reverse transcriptase activity, wherein the incubation permits reverse transcription of the RNA template.

In another aspect, a method for amplifying an RNA is disclosed, comprising incubating the RNA template in a reaction mixture comprising a mutant Archaeal DNA polymerase exhibiting an increased reverse transcriptase activity, wherein the incubation permits amplification of the RNA template.

In another aspect, a method for amplifying an RNA is disclosed, comprising: (a) incubating the RNA template in a first reaction mixture comprising a mutant Archaeal DNA polymerase exhibiting an increased reverse transcriptase activity, wherein the incubation permits reverse transcription of the RNA template to generate a cDNA template; and (b) incubating the cDNA template in a second reaction mixture, wherein that incubating permits amplification of the cDNA template.

In one embodiment, the second reaction mixture comprises a second DNA polymerase or a combination of two or more other DNA polymerases. In another embodiment, the second DNA polymerase is a wild-type DNA polymerase. In another embodiment, the second DNA polymerase comprises Taq DNA polymerase, Pfu Turbo DNA polymerase or a combination of these two.

### BRIEF DESCRIPTION OF THE DRAWINGS

FIG. 1 shows the primer sequences used for Pfu or JDF-3 mutagenesis according to some embodiments of the present invention.

FIG. 2 shows a comparison of RNA dependent DNA polymerization (reverse-transcriptase, RT) activity and DNA dependent DNA polymerase (DNA polymerase) activity in clarified lysates of wild-type and mutant Pfu and JDF-3 DNA polymerases. Three different volumes of clarified lysate were used for each polymerase. Top panel, DNA dependent DNA polymerase activity, measured as cpm of ³H-TTP incorporated; middle panel, RNA dependent DNA polymerase activity, measured as cpm of ³H-TTP incorporated; and bottom panel, ratios of RNA dependent polymerase activity over DNA polymerase activity from the samples with 0.2 μl of clarified lysate.

FIG. 3 shows a comparison of RNA dependent DNA polymerase activity and DNA dependent DNA polymerase activity in clarified lysates of Exo+ wild-type and mutant Pfu and JDF-3 DNA polymerases. Three different volumes of clarified lysate were used for each polymerase. Top panel, DNA dependent DNA polymerase activity, measured as cpm of ³H-TTP incorporated; middle panel, RNA dependent DNA polymerase activity, measured as cpm of ³H-TTP incorporated; and bottom panel, ratios of RNA dependent polymerase activity over DNA polymerase activity from the samples with 0.2 μl of clarified lysate.

FIG. 4 shows the results of experiments evaluating the reverse transcriptase activity of purified mutant polymerases according to several embodiments of the invention. Reactions were performed with purified preparations of exo- JDF-3 L408H and L408F mutants and with wild-type JDF-3 and Pfu and RNaseH⁻ MMLV-RT (Stratascript™, Stratagene). Activity is measured as Cpm of ³³P-dGTP incorporated. Improved RNA dependent DNA polymerase activity with the mutant polymerases is evident compared to wild type JDF-3 and Pfu.

FIG. 5 shows the results of an experiment evaluating the RNA dependent DNA polymerase activity of purified polymerase mutants by RT-PCR. A different purified polymerase (2 units) was used for each RT reaction, and Taq polymerase was used for subsequent PCR amplification. Products were separated by agarose gel electrophoresis and stained with ethidium bromide. Lane 1, negative control (no RTase); Lane 2, positive control using StrataScript™ RTase (RNaseH MMLV-RT); Lane 3, exo⁻ JDF-3 polymerase; Lane 4, exo⁻ JDF-3 L408H polymerase; and Lane 5, exo-JDF-3 L408F polymerase.

FIG. 6 is a sequence alignment of several Family B DNA polymerases. Pfu, Pyrococcus furiosus; JDF-3; Tgo, Thermococcus gorgonarius; Tli, Thermococcus litoralis; Tsp, Thermococcus sp.; Mvo, Methanococcus voltae; RB69, bacteriophage RB69; T4, bacteriophage T4; Eco, Eschericia coli. DNA polymerase sequences from additional species are aligned in Hopfner et al., 1999, Proc. Natl. Acad. Sci. U.S.A. 96: 3600-3605, which is incorporated herein by reference.

FIG. 7 contains the wild-type amino acid and polynucleotide sequences of representative Archaeal DNA polymerases, including JDF-3 DNA polymerase (SEQ ID NO: 1 and 2, respectively; amino acid sequence in the processed polypeptide is shown in italics, amino acids targeted for mutation according to several embodiments of the invention are underlined), wild type Pfu DNA polymerase (SEQ ID NO: 3 and 4, respectively), wild type KOD polymerase (SEQ ID NO: 5 and 6, respectively), wild type Vent™ polymerase (SEQ ID NO: 7 and 8, respectively), wild-type Deep Vent polymerase (SEQ ID NO: 9 and 10, respectively), Tgo DNA polymerase (SEQ ID NO: 11 and 12, respectively), Thest Thermococcus strain TY DNA polymerase (SEQ ID NO: 13 and 14, respectively), and 9oN Thermococcus species DNA polymerase (SEQ IDF NO: 15 and 16, respectively).

FIG. 8 shows data from an experiment evaluating the effect of DMSO concentration on the reverse transcriptase activity of the exo+ Pful409Y DNA polymerase mutant. M=RNA size markers. Lanes marked 0-25 correspond to reactions run in the presence of 0-25% DMSO.

### DETAILED DESCRIPTION

Definitions

As used herein, “polynucleotide polymerase” refers to an enzyme that catalyzes the polymerization of nucleotides, e.g., to synthesize polynucleotide strands from ribonucleoside triphosphates or deoxynucleoside triphosphates. Generally, the enzyme will initiate synthesis at the 3′-end of a primer annealed to a polynucleotide template sequence, and will proceed toward the 5′ end of the template strand. “DNA polymerase” catalyzes the polymerization of deoxynucleotides to synthesize DNA, while “RNA polymerase” catalyzes the polymerization of ribonucleotides to synthesize RNA.

The term “DNA polymerase” refers to a DNA polymerase which synthesizes new DNA strands by the incorporation of deoxynucleoside triphosphates in a template dependent manner. The measurement of DNA polymerase activity may be performed according to assays known in the art, for example, as described by a previously published method (Hogrefe, H. H., et al (01) Methods in Enzymology, 343:91-116). A “DNA polymerase” may be DNA-dependent (i.e., using a DNA template) or RNA-dependent (i.e., using a RNA template).

As used herein, the term “template dependent manner” refers to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, “thermostable” refers to a property of an enzyme that is active at elevated temperatures and is resistant to DNA duplex-denaturing temperatures in the range of about 93° C. to about 97° C. “Active” means the enzyme retains the ability to effect primer extension reactions when subjected to elevated or denaturing temperatures for the time necessary to effect denaturation of double-stranded nucleic acids. Elevated temperatures as used herein refer to the range of about 70° C. to about 75° C., whereas non-elevated temperatures as used herein refer to the range of about 35° C. to about 50° C.

As used herein, “Archaeal” refers to an organism or to a DNA polymerase from an organism of the kingdom Archaea, e.g., Archaebacteria. An “Archaeal DNA polymerase” refers to any identified or unidentified DNA polymerase (e.g., as described in Tables II-IV) isolated from an Archaeabacteria, e.g., as described in Table V.

As used herein, the term “reverse transcriptase (RT)” describes a class of polymerases characterized as RNA dependent DNA polymerases. RT is a critical enzyme responsible for the synthesis of cDNA from viral RNA for all retroviruses, including HIV, HTLV-I, HTLV-II, FeLV, FIV, SIV, AMV, MMTV, and MoMuLV. For review, see e.g. Levin, 1997, Cell, 88:5-8; Brosius et al., 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

As used herein, the terms “reverse transcription activity” and “reverse transcriptase activity” are used interchangeably to refer to the ability of an enzyme (e.g., a reverse transcriptase or a DNA polymerase) to synthesize a DNA strand (i.e., cDNA) utilizing an RNA strand as a template. Methods for measuring RT activity are provided in the examples herein below and also are well known in the art. For example, the Quan-T-RT assay system is commercially available from Amersham (Arlington Heights, Ill.) and is described in Bosworth, et al., Nature 1989, 341:167-168.

As used herein, the term “increased reverse transcriptase activity” refers to the level of reverse transcriptase activity of a mutant enzyme (e.g., a DNA polymerase) as compared to its wild-type form. A mutant enzyme is said to have an “increased reverse transcriptase activity” if the level of its reverse transcriptase activity (as measured by methods described herein or known in the art) is at least 20% or more than its wild-type form, for example, at least 25%, 30%, 40%, 50%, 60%, 70%, 80%, 90%, 100% more or at least 2-fold, 3-fold, 4-fold, 5-fold, or 10-fold or more.

As used herein, “exonuclease” refers to an enzyme that cleaves bonds, preferably phosphodiester bonds, between nucleotides one at a time from the end of a DNA molecule. An exonuclease can be specific for the 5′ or 3′ end of a DNA molecule, and is referred to herein as a 5′ to 3′ exonuclease or a 3′ to 5′ exonuclease. The 3′ to 5′ exonuclease degrades DNA by cleaving successive nucleotides from the 3′ end of the polynucleotide while the 5′ to 3′ exonuclease degrades DNA by cleaving successive nucleotides from the 5′ end of the polynucleotide. During the synthesis or amplification of a polynucleotide template, a DNA polymerase with 3′ to 5′ exonuclease activity (3′ to 5′ exo⁺) has the capacity of removing mispaired base (proofreading activity), therefore is less error-prone (i.e., with higher fidelity) than a DNA polymerase without 3′ to 5′ exonuclease activity (3′ to 5′ exo⁻). The exonuclease activity can be measured by methods well known in the art. For example, one unit of exonuclease activity may refer to the amount of enzyme required to cleave 1 μg DNA target in an hour at 37° C.

The term “substantially free of 5′ to 3′ exonuclease activity” indicates that the enzyme has less than about 5% of the 5′ to 3′ exonuclease activity of wild-type enzyme, preferably less than about 3% of the 5′ to 3′ exonuclease activity of wild-type enzyme, and most preferably no detectable 5′ to 3′ exonuclease activity. The term “substantially free of 3′ to 5′ exonuclease activity” indicates that the enzyme has less than about 5% of the 3′ to 5′ exonuclease activity of wild-type enzyme, preferably less than about 3% of the 3′ to 5′ exonuclease activity of wild-type enzyme, and most preferably no detectable 3′ to 5′ exonuclease activity.

The term “fidelity” as used herein refers to the accuracy of DNA polymerization by template-dependent DNA polymerase, e.g., RNA-dependent or DNA-dependent DNA polymerase. The fidelity of a DNA polymerase is measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity of a DNA polymerase. The term “high fidelity” refers to an error rate of 5×10⁻⁶ per base pair or lower. The fidelity or error rate of a DNA polymerase may be measured using assays known to the art (see for example, Lundburg et al., 1991 Gene, 108:1-6).

As used herein, an “amplified product” refers to the single- or double-strand polynucleotide population at the end of an amplification reaction. The amplified product contains the original polynucleotide template and polynucleotide synthesized by DNA polymerase using the polynucleotide template during the amplification reaction.

As used herein, “polynucleotide template” or “target polynucleotide template” refers to a polynucleotide (RNA or DNA) which serves as a template for a DNA polymerase to synthesize DNA in a template-dependent manner. The “amplified region,” as used herein, is a region of a polynucleotide that is to be either synthesized by reverse transcription or amplified by polymerase chain reaction (PCR). For example, an amplified region of a polynucleotide template may reside between two sequences to which two PCR primers are complementary to.

As used herein, “primer” refers to an oligonucleotide, whether natural or synthetic, which is substantially complementary to a template DNA or RNA (i.e., at least 7 out of 10, preferably 9 out of 10, more preferably 9 out of 10 bases are fully complementary) and can anneal to a complementary template DNA or RNA to form a duplex between the primer and the template. A primer may serve as a point of initiation of nucleic acid synthesis by a polymerase following annealing to a DNA or RNA strand. A primer is typically a single-stranded oligodeoxyribonucleotide. The appropriate length of a primer depends on the intended use of the primer, typically ranges from about 10 to about 60 nucleotides in length, preferably 15 to 40 nucleotides in length.

“Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue.

As used herein in relation to the position of an amino acid mutation, the term “corresponding to” refers to an amino acid in a first polypeptide sequence that aligns with a given amino acid in a reference polypeptide sequence when the first polypeptide and reference polypeptide sequences are aligned. Alignment is performed by one of skill in the art using software designed for this purpose, for example, BLASTP version 2.2.2 with the default parameters for that version. As an example of amino acids that “correspond,” L408 of the JDF-3 Family B DNA polymerase of SEQ ID NO: 1 “corresponds to” L409 of Pfu DNA polymerase, and vice versa, and L409 of Pfu DNA polymerase “corresponds to” L454 of Methanococcus voltae DNA polymerase and vice versa.

The term “wild-type” refers to a gene or gene product which has the characteristics of that gene or gene product when isolated from a naturally occurring source. In contrast, the term “modified” or “mutant” refers to a gene or gene product which displays altered nucleotide or amino acid sequence(s) (i.e., mutations) when compared to the wild-type gene or gene product. For example, a mutant enzyme in the present invention is a mutant DNA polymerase which exhibits an increased reverse transcriptase activity, compared to its wild-type form.

As used herein, the term “mutation” refers to a change in nucleotide or amino acid sequence within a gene or a gene product, or outside the gene in a regulatory sequence compared to wild type. The change may be a deletion, substitution, point mutation, mutation of multiple nucleotides or amino acids, transposition, inversion, frame shift, nonsense mutation or other forms of aberration that differentiate the polynucleotide or protein sequence from that of a wild-type sequence of a gene or a gene product.

As used herein, the term “polynucleotide binding protein” refers to a protein which is capable of binding to a polynucleotide. A useful polynucleotide binding protein according to the present invention includes, but is not limited to: Ncp7, recA, SSB, T4gp32, an Archaeal sequence non-specific double stranded DNA binding protein (e.g., Sso7d, Sac7d, PCNA (WO 01/92501, incorporated herein by reference)), and a helix-hairpin-helix domain.

As used herein, the term “Archaeal accessory factor” refers to a polypeptide factor that enhances the reverse transcriptase or polymerase activity of an Archaeal DNA polymerase. The accessory factor can enhance the fidelity and/or processivity of the DNA polymerase or reverse transcriptase activity of the enzyme. Non-limiting examples of Archaeal accessory factors are provided in WO 01/09347, and U.S. Pat. No. 6,333,158 which are incorporated herein by reference.

As used herein, the term “vector” refers to a polynucleotide used for introducing exogenous or endogenous polynucleotide into host cells. A vector comprises a nucleotide sequence which may encode one or more polypeptide molecules. Plasmids, cosmids, viruses and bacteriophages, in a natural state or which have undergone recombinant engineering, are non-limiting examples of commonly used vectors to provide recombinant vectors comprising at least one desired isolated polynucleotide molecule.

As used herein, the term “transformation” or the term “transfection” refers to a variety of art-recognized techniques for introducing exogenous polynucleotide (e.g., DNA) into a cell. A cell is “transformed” or “transfected” when exogenous DNA has been introduced inside the cell membrane. The terms “transformation” and “transfection” and terms derived from each are used interchangeably.

As used herein, an “expression vector” refers to a recombinant expression cassette which has a polynucleotide which encodes a polypeptide (i.e., a protein) that can be transcribed and translated by a cell. The expression vector can be a plasmid, virus, or polynucleotide fragment.

As used herein, “isolated” or “purified” when used in reference to a polynucleotide or a polypeptide means that a naturally occurring nucleotide or amino acid sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the nucleotide or amino acid sequence is the only polynucleotide or polypeptide present, but that it is essentially free (about 90-95%, up to 99-100% pure) of non-polynucleotide or polypeptide material naturally associated with it.

As used herein the term “encoding” refers to the inherent property of specific sequences of nucleotides in a polynucleotide, such as a gene in a chromosome or an mRNA, to serve as templates for synthesis of other polymers and macromolecules in biological processes having a defined sequence of nucleotides (i.e., rRNA, tRNA, other RNA molecules) or amino acids and the biological properties resulting therefrom. Thus a gene encodes a protein, if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. A polynucleotide that encodes a protein includes any polynucleotides that have different nucleotide sequences but encode the same amino acid sequence of the protein due to the degeneracy of the genetic code.

Amino acid residues identified herein are preferred in the natural L-configuration. In keeping with standard polypeptide nomenclature, J. Biol. Chem., 243:3557-3559, 1969, abbreviations for amino acid residues are as shown in the following Table I.

TABLE I

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

The invention relates to the discovery of thermostable DNA polymerases, e.g., Archaeal DNA polymerases, that bear one or more mutations resulting in increased reverse transcriptase activity relative to their unmodified wild-type forms. All references described herein are incorporated by reference herein in their entirety.

Thermostable DNA Polymerases

Reverse transcription from many RNA templates by commonly used reverse transcriptases such as avian myeloblastosis virus (AMV) reverse transcriptase and Moloney murine leukemia virus (MMLV) reverse transcriptase is often limited by the secondary structure of the RNA template. Secondary structure in RNA results from hybridization between complementary regions within a given RNA molecule. Secondary structure causes poor synthesis of cDNA and premature termination of CDNA products because polymerases are unable to process through the secondary structure. Therefore, RNAs with secondary structure may be poorly represented in a cDNA library and detection of the presence of RNA with secondary structure in a sample by RT-PCR may be difficult. Furthermore, secondary structure in RNA may cause inconsistent results in techniques such as differential display PCR. Accordingly, it is advantageous to conduct reverse transcription reactions at increased temperatures so that secondary structure is removed or limited.

Several thermostable eubacterial DNA polymerases (e.g., T. thermophilus DNA polymerase, T. aquaticus DNA polymerase (e.g., U.S. Pat. No. 5,322,770), A. thermophilum DNA polymerase (e.g., WO 98/14588), T. vulgaris DNA polymerase (e.g., U.S. Pat. No. 6,436,677), B. caldotenax DNA polymerase (e.g., U.S. Pat. No. 5,436,149); and the polymerase mixture marketed as C. THERM (Boehringer Mannheim) have been demonstrated to possess reverse transcriptase activity. These enzymes can be used at higher temperatures than retroviral reverse transcriptases so that much of the secondary structure of RNA molecules is removed.

The present invention provides a thermostable archacal DNA polymerase with increased reverse transcriptase activity. A wild-type thermostable DNA polymerase useful for the present invention may or may not possess native reverse transcriptase activity. Useful wild-type thermostable DNA polymerases according to the present invention include, but are not limited to, the polymerases listed in Tables II and III.

In one embodiment, a wild-type Archaeal DNA polymerase is used to produce a thermostable DNA polymerase with increased reverse transcriptase activity.

Thermostable Archaeal DNA polymerases are typically isolated from Archeobacteria. Archeobacterial organisms from which Archaeal DNA polymerases useful in the present invention may be obtained are shown, but not limited to the species shown, in Table IV. The Archaebacteria include a group of “hyperthermophiles” that grow optimally around 100° C. These organisms grow at temperatures higher than 90^{□} C. and their enzymes demonstrate greater themostability (Mathur et al., 1992, Stratagies 5:11) than the thermophilic eubacterial DNA polymerases. They are presently represented by three distinct genera, Pyrodictium, Pyrococcus, and Pyrobaculum. Pryodictium brockii (Tₒₚₜ 105° C.) is an obligate autotroph which obtains energy be reducing S^{o} to H₂S^{o} with H₂, while Pyrobaculum islandicum (Tₒₚₜ 100° C.) is a faculative heterotroph that uses either organic substrates or H₂ to reduce S^{o}. In contrast, Pyrococcus furiosus (Tₒₚₜ 100° C.) grows by a fermentative-type metabolism rather than by S^{o} respiration. It is a strict heterotroph that utilizes both simple and complex carbohydrates where only H₂ and CO₂ are the detectable products. The organism reduces elemental sulfur to H₂S apparently as a form of detoxification since H₂ inhibits growth.

TABLE II
ARCHAEAL FAMILY B DNA POLYMERASES*

| DNA polymerase | Citation |
|---|---|
| Thermococcus litoralis DNA polymerase (Vent) | Perler et al., (1992) Proc. Natl. |
| | Acad. Sci. USA 89, 5577-5581. |
| | Kong et al., J. Biol. Chem. |
| | 268: 1965 (1993) |
| | U.S. Pat. No. 5,210,036 |
| | U.S. Pat. No. 5,322,785 |
| Pyrococcus sp. DNA polymerase (Deep Vent, | Xu et al., Cell 75 (7), 1371-1377 |
| from Pyrococcus sp. GB-D) | (1993) |
| Pyrococcus furiosus DNA polymerase | Mathur et al., (1991) Nucleic. |
| | Acids Res. 19, 6952. |
| | Lundberg et al., Gene 108:1 |
| | (1991) |
| | PCT Pub. WO 92/09689 |
| | U.S. Pat. No. 5,948,663 |
| | U.S. Pat. No. 5,866,395 |
| Sulfolobus solfataricus DNA polymerase | Pisani et al., (1992) Nucl. |
| | Acids Res. 20, 2711-2716. |
| Thermococcus gorgonarius DNA polymerase | Hopfner, K.P. et al. (1999) |
| | Proc. Natl. Acad. Sci. U.S.A. |
| | 96, 3600-3605. |
| Thermococcus species TY | Niehaus, F. et al. (1997) Gene |
| | 204, 153-158. |
| Thermococcus species strain KODI (formerly | Tagaki et al. (1997) Appl. |
| classified as Pyrococcus sp.) | Environ. Microbiol. 63, 4504-4510. |
| | U.S. Pat. No. 6,008,025 |
| JDF-3 DNA polymerase | U.S. Pat. Nos. 5,602,011; |
| | 5,948,663; 5,866,395; |
| | 5,545,552; 5,556,772 |
| Sulfolobus acidocaldarius | Datukishvili, N. et al. (1996) |
| | Gene 177, 271-273. |
| | Salhi et al., J. Mol. Biol., |
| | 209: 635-641 (1989). |
| | Salhi et al., Biochem. Biophys. |
| | Res. Comm., 167: 1341-1347 |
| | (1990). |
| | Rella et al., Ital. J. Biochem., |
| | 39: 83-99 (1990). |
| | Forterre et al., Can. J. |
| | Microbiol., 35: 228-233 (1989). |
| | Rossi et al., System. Appl. |
| | Microbiol., 7: 337-341 (1986). |
| | Klimczak et al., Nucleic Acids |
| | Res., 13: 5269-5282 (1985). |
| | Elie et al., Biochim. Biophys. |
| | Acta 951: 261-267 (1988). |
| Thermococcus species 9° N-7 | Southworth, M.W. et al. |
| | (1996) Proc. Natl. Acad. Sci. |
| | U.S.A. 93, 5281-5285. |
| Pyrodictium occultum | Uemori, T. et al. (1995) J. |
| | Bacteriol. 177, 2164-2177. |
| Methanococcus voltae | Konisky, J. et al. (1994) J. |
| | Bacteriol. 176, 6402-6403. |
| Desulfurococcus strain TOK (D. Tok Pol) | Zhao (1999) Structure Fold |
| | Des. 7, 1189. |

*All members have an LYP motif in Region II at a position corresponding to L408 of JDF-3 Family B DNA polymerase of SEQ ID NO: 1.

TABLE III
ACCESSION INFORMATION FOR CERTAIN
THERMOSTABLE DNA POLYMERASES
Vent Thermococcus litoralis

| | |
|---|---|
| ACCESSION | AAA72101 |
| PID | g348689 |
| VERSION | AAA72101.1 GI: 348689 |
| DBSOURCE | locus THCVDPE accession M74198.1 |

Thest Thermococcus Sp. (Strain Ty)

| | |
|---|---|
| ACCESSION | O33845 |
| PID | g3913524 |
| VERSION | O33845 GI: 3913524 |
| DBSOURCE | swissprot: locus DPOL_THEST, accession O33845 |

Pab Pyrococcus abyssi

| | |
|---|---|
| ACCESSION | P77916 |
| PID | g3913529 |
| VERSION | P77916 GI: 3913529 |
| DBSOURCE | swissprot: locus DPOL_PYRAB, accession P77916 |
| PYRHO | Pyrococcus horikoshii |
| ACCESSION | O59610 |
| PID | g3913526 |
| VERSION | O59610 GI: 3913526 |
| DBSOURCE | swissprot: locus DPOL_PYRHO, accession O59610 |

Pyrse Pyrococcus Sp. (Strain Ge23)

| | |
|---|---|
| ACCESSION | P77932 |
| PID | g3913530 |
| VERSION | P77932 GI: 3913530 |
| DBSOURCE | swissprot: locus DPOL_PYRSE, accession P77932 |

DeepVent Pyrococcus sp.

| | |
|---|---|
| ACCESSION | AAA67131 |
| PID | g436495 |
| VERSION | AAA67131.1 GI: 436495 |
| DBSOURCE | locus PSU00707 accession U00707.1 |

Pfu Pyrococcus furiosus

| | |
|---|---|
| ACCESSION | P80061 |
| PID | g399403 |
| VERSION | P80061 GI: 399403 |
| DBSOURCE | swissprot: locus DPOL_PYRFU, accession P80061 |
| JDF-3 | Thermococcus sp. |
| ACCESSION | AX135459 |

Baross gi|2097756|pat. |U.S. |No. 5602011|12 Sequence
12 from U.S. Pat. No. 5602011

9° N Thermococcus Sp. (Strain 9° N −7).

| | |
|---|---|
| ACCESSION | Q56366 |
| PID | g3913540 |
| VERSION | Q56366 GI: 3913540 |
| DBSOURCE | swissprot: locus DPOL_THES9, accession Q56366 |
| KOD | Pyrococcus sp. |
| ACCESSION | BAA06142 |
| PID | g1620911 |
| VERSION | BAA06142.1 GI: 1620911 |
| DBSOURCE | locus PYWKODPOL accession D29671.1 |

Tgo Thermococcus gorgonarius.

| | |
|---|---|
| ACCESSION | 4699806 |
| PID | g4699806 |
| VERSION | GI: 4699806 |
| DBSOURCE | pdb: chain 65, release Feb. 23, 1999 |
| THEFM | Thermococcus fumicolans |
| ACCESSION | P74918 |
| PID | g3913528 |
| VERSION | P74918 GI: 3913528 |
| DBSOURCE | swissprot: locus DPOL_THEFM, accession P74918 |

METTH Methanobacterium thermoautotrophicum

| | |
|---|---|
| ACCESSION | O27276 |
| PID | g3913522 |
| VERSION | O27276 GI: 3913522 |
| DBSOURCE | swissprot: locus DPOL_METTH, accession |
| | O27276 |

Methanococcus jannaschii

| | |
|---|---|
| ACCESSION | Q58295 |
| PID | g3915679 |
| VERSION | Q58295 GI: 3915679 |
| DBSOURCE | swissprot: locus DPOL_METJA, accession Q58295 |
| POC | Pyrodictium occultum |
| ACCESSION | B56277 |
| PID | g1363344 |
| VERSION | B56277 GI: 1363344 |
| DBSOURCE | pir: locus B56277 |

ApeI Aeropyrum pernix

| | |
|---|---|
| ACCESSION | BAA81109 |
| PID | g5105797 |
| VERSION | BAA81109.1 GI: 5105797 |
| DBSOURCE | locus AP000063 accession AP000063.1 |
| ARCFU | Archaeoglobus fulgidus |
| ACCESSION | O29753 |
| PID | g3122019 |
| VERSION | O29753 GI: 3122019 |
| DBSOURCE | swissprot: locus DPOL_ARCFU, accession O29753 |

Desulfurococcus sp. Tok.

| | |
|---|---|
| ACCESSION | 6435708 |
| PID | g64357089 |
| VERSION | GT: 6435708 |
| DBSOURCE | pdb. chain 65, release Jun. 2, 1999 |

TABLE IV
CRENARCHAEOTA (EXTREMELY THERMOPHILIC
ARCHAEBACTERIA)

```
□ Thermoprotei
  □ Desulfurococcales
    □ Desulfurococcaceae
      □ Aeropyrum
        ▪ Aeropyrum pernix
      □ Desulfurococcus
        ▪ Desulfurococcus amylolyticus
        ▪ Desulfurococcus mobilis
        ▪ Desulfurococcus mucosus
        ▪ Desulfurococcus saccharovorans
        ▪ Desulfurococcus sp.
        ▪ Desulfurococcus sp. SEA
        ▪ Desulfurococcus sp. SY
        ▪ Desulfurococcus sp. Tok
      □ Ignicoccus
        ▪ Ignicoccus islandicus
        ▪ Ignicoccus pacificus
      □ Staphylothermus
        ▪ Staphylothermus hellenicus
        ▪ Staphylothermus marinus
      □ Stetteria
        ▪ Stetteria hydrogenophila
      □ Sulfophobococcus
        ▪ Sulfophobococcus zilligii
      □ Thermodiscus
        ▪ Thermodiscus maritimus
      □ Thermosphaera
        ▪ Thermosphaera aggregans
    □ Pyrodictiaceae
      □ Hyperthermus
        ▪ Hyperthermus butylicus
      □ Pyrodictium
        ▪ Pyrodictium abyssi
        ▪ Pyrodictium brockii
        ▪ Pyrodictium occultum
      □ Pyrolobus
        ▪ Pyrolobus fumarii
    □ unclassified Desulfurococcales
      □ Acidilobus
        ▪ Acidilobus aceticus
      □ Caldococcus
        ▪ Caldococcus noboribetus
  □ Sulfolobales
    □ Sulfolobaceae
      □ Acidianus
        ▪ Acidianus ambivalens
        ▪ Acidianus brierleyi
        ▪ Acidianus infernus
        ▪ Acidianus sp. S5
      □ Metallosphaera
        ▪ Metallosphaera prunae
        ▪ Metallosphaera sedula
        ▪ Metallosphaera sp.
        ▪ Metallosphaera sp. GIB11/00
        ▪ Metallosphaera sp. J1
      □ Stygiolobus
        ▪ Stygiolobus azoricus
      □ Sulfolobus
        ▪ Sulfolobus acidocaldarius
        ▪ Sulfolobus islandicus
        ▪ Sulfolobus metallicus
        ▪ Sulfolobus shibatae
        ▪ Sulfolobus solfataricus
        ▪ Sulfolobus thuringiensis
        ▪ Sulfolobus tokodaii
        ▪ Sulfolobus yangmingensis
        ▪ Sulfolobus sp.
        ▪ Sulfolobus sp. AMP12/99
        ▪ Sulfolobus sp. CH7/99
        ▪ Sulfolobus sp. FF5/00
        ▪ Sulfolobus sp. MV2/99
        ▪ Sulfolobus sp. MVSoil3/SC2
        ▪ Sulfolobus sp. MVSoil6/SC1
        ▪ Sulfolobus sp. NGB23/00
        ▪ Sulfolobus sp. NGB6/00
        ▪ Sulfolobus sp. NL8/00
        ▪ Sulfolobus sp. NOB8H2
        ▪ Sulfolobus sp. RC3
        ▪ Sulfolobus sp. RC6/00
        ▪ Sulfolobus sp. RCSC1/01
      □ Sulfurisphaera
        ▪ Sulfurisphaera ohwakuensis
  □ Thermoproteales
    □ Thermofiliaceae
      □ Thermofilum
        ▪ Thermofilum librum
        ▪ Thermofilum pendens
      □ unclassified Thermofiliaceae
        ▪ Thermofiliaceae str. SRI-325
        ▪ Thermofiliaceae str. SRI-370
    □ Thermoproteaceae
      □ Caldivirga
        ▪ Caldivirga maquilingensis
      □ Pyrobaculum
        □ Pyrobaculum aerophilum
        ▪ Pyrobaculum arsenaticum
        ▪ Pyrobaculum islandicum
        ▪ Pyrobaculum neutrophilum
        ▪ Pyrobaculum oguniense
        ▪ Pyrobaculum organotrophum
        ▪ Pyrobaculum sp. WIJ3
      □ Thermocladium
        ▪ Thermocladium modestius
      □ Thermoproteus
        ▪ Thermoproteus neutrophilus
        ▪ Thermoproteus tenax
        ▪ Thermoproteus sp. IC-033
        ▪ Thermoproteus sp. IC-061
      □ Vulcanisaeta
        ▪ Vulcanisaeta distributa
        ▪ Vulcanisaeta souniana
□ Euryarchaeota
  □ Archaeoglobi
  □ Archaeoglobales
    □ Archaeoglobaceae
      □ Archaeoglobus
        ▪ Archaeoglobus fulgidus
        ▪ Archaeoglobus lithotrophicus
        ▪ Archaeoglobus profundus
        ▪ Archaeoglobus veneficus
      □ Ferroglobus
        ▪ Ferroglobus placidus
□ Halobacteria
  □ Halobacteriales
    □ Halobacteriaceae
      □ Haloalcalophilium
        ▪ Haloalcalophilium atacamensis
      □ Haloarcula
        ▪ Haloarcula aidinensis
        ▪ Haloarcula argentinensis
        ▪ Haloarcula hispanica
        ▪ Haloarcula japonica
        □ Haloarcula marismortui
          ▪ Haloarcula marismortui subsp.
          marismortui
        ▪ Haloarcula mukohataei
        ▪ Haloarcula sinaiiensis
        ▪ Haloarcula vallismortis
        ▪ Haloarcula sp.
        ▪ Haloarcula sp. ARG-2
    □ Halobacterium
      □ Halobacterium salinarum
          ▪ Halobacterium salinarum (strain Mex)
          ▪ Halobacterium salinarum (strain Port)
          ▪ Halobacterium salinarum (strain Shark)
        ▪ Halobacterium sp.
        ▪ Halobacterium sp. 9R
        ▪ Halobacterium sp. arg-4
        ▪ Halobacterium sp. AUS-1
        ▪ Halobacterium sp. AUS-2
        ▪ Halobacterium sp. GRB
        ▪ Halobacterium sp. JP-6
        ▪ Halobacterium sp. NCIMB 714
        ▪ Halobacterium sp. NCIMB 718
        ▪ Halobacterium sp. NCIMB 720
        ▪ Halobacterium sp. NCIMB 733
        ▪ Halobacterium sp. NCIMB 734
        ▪ Halobacterium sp. NCIMB 741
        ▪ Halobacterium sp. NCIMB 765
        ▪ Halobacterium sp. NRC-1
        ▪ Halobacterium sp. NRC-817
        ▪ Halobacterium sp. SG1
      □ Halobaculum
        ▪ Halobaculum gomorrense
      □ Halococcus
        ▪ Halococcus dombrowskii
        ▪ Halococcus morrhuae
        ▪ Halococcus saccharolyticus
        ▪ Halococcus salifodinae
        ▪ Halococcus tibetense
        ▪ Halococcus sp.
      □ Haloferax
        ▪ Haloferax alexandrinus
        ▪ Haloferax alicantei
        ▪ Haloferax denitrificans
        ▪ Haloferax gibbonsii
        ▪ Haloferax mediterranei
        ▪ Haloferax volcanii
        ▪ Haloferax sp.
        ▪ Haloferax sp. D1227
        ▪ Haloferax sp. LWp2.1
      □ Halogeometricum
        ▪ Halogeometricum borin quense
      □ Halorhabdus
        ▪ Halorhabdus utahensis
      □ Halorubrum
        ▪ Halorubrum coriense
        ▪ Halorubrum distributum
        ▪ Halorubrum lacusprofundi
        ▪ Halorubrum saccharovorum
        ▪ Halorubrum sodomense
        ▪ Halorubrum tebenquichense
        ▪ Halorubrum tibetense
        ▪ Halorubrum trapanicum
        ▪ Halorubrum vacuolatum
        ▪ Halorubrum sp. GSL5.48
        ▪ Halorubrum sp. SC1.2
      □ Halosimplex
        ▪ Halosimplex carlsbadense
      □ Haloterrigena
        ▪ Haloterrigena thermotolerans
        ▪ Haloterrigena turkmenicus
      □ Natrialba
        ▪ Natrialba aegyptia
        ▪ Natrialba asiatica
        ▪ Natrialba chahannaoensis
        ▪ Natrialba hulunbeirensis
        ▪ Natrialba magadii
        ▪ Natrialba sp. ATCC 43988
        ▪ Natrialba sp. Tunisia HMg-25
        ▪ Natrialba sp. Tunisia HMg-27
      □ Natrinema
        ▪ Natrinema versiforme
        ▪ Natrinema sp. R-fish
      □ Natronobacterium
        ▪ Natronobacterium gregoryi
        ▪ Natronobacterium innermongoliae
        ▪ Natronobacterium nitratireducens
        ▪ Natronobacterium wudunaoensis
      □ Natronococcus
        ▪ Natronococcus amylolyticus
        ▪ Natronococcus occultus
        ▪ Natronococcus xinjiangense
        ▪ Natronococcus sp.
      □ Natronomonas
        ▪ Natronomonas pharaonis
      □ Natronorubrum
        ▪ Natronorubrum bangense
        ▪ Natronorubrum tibetense
        ▪ Natronorubrum sp. Tenzan-10
        ▪ Natronorubrum sp. Wadi Natrun-19
□ Methanobacteria
  □ Methanobacteriales
    □ Methanobacteriaceae
      □ Methanobacterium
        ▪ Methanobacterium bryantii
        ▪ Methanobacterium congolense
        ▪ Methanobacterium curvum
        ▪ Methanobacterium defluvii
        ▪ Methanobacterium espanolae
        ▪ Methanobacterium formicicum
        ▪ Methanobacterium ivanovii
        ▪ Methanobacterium oryzae
        ▪ Methanobacterium palustre
        ▪ Methanobacterium subterraneum
        ▪ Methanobacterium thermaggregans
        ▪ Methanobacterium thermoflexum
        ▪ Methanobacterium thermophilum
        ▪ Methanobacterium uliginosum
        ▪ Methanobacterium sp.
      □ Methanobrevibacter
        ▪ Methanobrevibacter arboriphilus
        ▪ Methanobrevibacter curvatus
        ▪ Methanobrevibacter cuticularis
        ▪ Methanobrevibacter filiformis
        ▪ Methanobrevibacter oralis
        ▪ Methanobrevibacter ruminantium
        ▪ Methanobrevibacter smithii
        ▪ methanogenic endosymbiont of Nyctotherus
        cordiformis
        ▪ methanogenic endosymbiont of Nyctotherus
        ovalis
        ▪ methanogenic endosymbiont of Nyctotherus
        velox
        ▪ methanogenic symbiont RS104
        ▪ methanogenic symbiont RS105
        ▪ methanogenic symbiont RS208
        ▪ methanogenic symbiont RS301
        ▪ methanogenic symbiont RS404
        ▪ Methanobrevibacter sp.
        ▪ Methanobrevibacter sp. ATM
        ▪ Methanobrevibacter sp. FMB1
        ▪ Methanobrevibacter sp. FMB2
        ▪ Methanobrevibacter sp. FMB3
        ▪ Methanobrevibacter sp. FMBK1
        ▪ Methanobrevibacter sp. FMBK2
        ▪ Methanobrevibacter sp. FMBK3
        ▪ Methanobrevibacter sp. FMBK4
        ▪ Methanobrevibacter sp. FMBK5
        ▪ Methanobrevibacter sp. FMBK6
        ▪ Methanobrevibacter sp. FMBK7
        ▪ Methanobrevibacter sp. HW23
        ▪ Methanobrevibacter sp. LRsD4
        ▪ Methanobrevibacter sp. MD101
        ▪ Methanobrevibacter sp. MD102
        ▪ Methanobrevibacter sp. MD103
        ▪ Methanobrevibacter sp. MD104
        ▪ Methanobrevibacter sp. MD105
        ▪ Methanobrevibacter sp. RsI3
        ▪ Methanobrevibacter sp. RsW3
        ▪ Methanobrevibacter sp. XT106
        ▪ Methanobrevibacter sp. XT108
        ▪ Methanobrevibacter sp. XT109
      □ Methanosphaera
        ▪ Methanosphaera stadtmanae
      □ Methanothermobacter
        □ Methanothermobacter marburgensis
        ▪ Methanothermobacter marburgensis str.
        Marburg
        □ Methanothermobacter thermautotrophicus
        ▪ Methanothermobacter
        thermautotrophicus str. Winter
        ▪ Methanothermobacter wolfeii
    □ Methanothermaceae
      □ Methanothermus
        ▪ Methanothermus fervidus
        ▪ Methanothermus sociabilis
  □ Methanococci
  □ Methanococcales
    □ Methanococcaceae
      □ Methanococcus
        ▪ Methanococcus aeolicus
        ▪ Methanococcus fervens
        ▪ Methanococcus igneus
        ▪ Methanococcus infernus
        ▪ Methanococcus jannaschii
        ▪ Methanococcus maripaludis
        ▪ Methanococcus vannielii
        ▪ Methanococcus voltae
        ▪ Methanococcus vulcanius
        ▪ Methanococcus sp. P2F9701a
      □ Methanothermococcus
        ▪ Methanothermococcus okinawensis
        ▪ Methanothermococcus thermolithotrophicus
  □ Methanomicrobiales
    □ Methanocorpusculaceae
      □ Methanocorpusculum
        ▪ Methanocorpusculum aggregans
        ▪ Methanocorpusculum bavaricum
        ▪ Methanocorpusculum labreanum
        ▪ Methanocorpusculum parvum
        ▪ Methanocorpusculum sinense
        ▪ Metopus contortus archaeal symbiont
        ▪ Metopus palaeformis endosymbiont
        ▪ Trimyema sp. archaeal symbiont
    □ Methanomicrobiaceae
      □ Methanocalculus
        ▪ Methanocalculus halotolerans
        ▪ Methanocalculus taiwanense
        ▪ Methanocalculus sp. K1F9705b
        ▪ Methanocalculus sp. K1F9705c
        ▪ Methanocalculus sp. O1F9702c
      □ Methanoculleus
        ▪ Methanoculleus bourgensis
        ▪ Methanoculleus chikugoensis
        ▪ Methanoculleus marisnigri
        ▪ Methanoculleus olentangyi
        ▪ Methanoculleus palmolei
        ▪ Methanoculleus thermophilicus
        ▪ Methanoculleus sp.
        ▪ Methanoculleus sp. BA1
        ▪ Methanoculleus sp. MAB1
        ▪ Methanoculleus sp. MAB2
        ▪ Methanoculleus sp. MAB3
      □ Methanofollis
        ▪ Methanofollis aquaemaris
        □ Methanofollis liminatans
        ▪ Methanofollis tationis
      □ Methanogenium
        ▪ Methanogenium cariaci
        ▪ Methanogenium frigidum
        ▪ Methanogenium organophilum
        ▪ Methanogenium sp.
      □ Methanomicrobium
        ▪ Methanomicrobium mobile
      □ Methanoplanus
        ▪ Methanoplanus endosymbiosus
        ▪ Methanoplanus limicola
        ▪ Methanoplanus petrolearius
      □ Methanospirillum
        ▪ Methanospirillum hungatei
        ▪ Methanospirillum sp.
  □ Methanosarcinales
    □ Methanosaetaceae
      □ Methanosaeta
        ▪ Methanosaeta concilii
        ▪ Methanothrix thermophila
        ▪ Methanosaeta sp.
        ▪ Methanosaeta sp. AMPB-Zg
    □ Methanosarcinaceae
      □ Methanimicrococcus
        ▪ Methanimicrococcus blatticola
      □ Methanococcoides
        ▪ Methanococcoides burtonii
        ▪ Methanococcoides methylutens
        ▪ Methanococcoides sp. NaT1
      □ Methanohalobium
        ▪ Methanohalobium evestigatum
        ▪ Methanohalobium sp. strain SD-1
      □ Methanohalophilus
        ▪ Methanohalophilus euhalobius
        ▪ Methanohalophilus halophilus
        ▪ Methanohalophilus mahii
        ▪ Methanohalophilus oregonensis
        ▪ Methanohalophilus portucalensis
        ▪ Methanohalophilus zhilinae
        ▪ Methanohalophilus sp. strain Cas-1
        ▪ Methanohalophilus sp. strain HCM6
        ▪ Methanohalophilus sp. strain Ref-1
        ▪ Methanohalophilus sp. strain SF-1
      □ Methanolobus
        ▪ Methanolobus bombayensis
        ▪ Methanolobus taylorii
        ▪ Methanolobus tindarius
        ▪ Methanolobus vulcani
      □ Methanomethylovorans
        ▪ Methanomethylovorans hollandica
        ▪ Methanomethylovorans victoriae
      □ Methanosarcina
        □ Methanosarcina acetivorans
        ▪ Methanosarcina barkeri
        ▪ Methanosarcina lacustris
        ▪ Methanosarcina mazei
        ▪ Methanosarcina semesiae
        ▪ Methanosarcina siciliae
        ▪ Methanosarcina thermophila
        ▪ Methanosarcina vacuolata
        ▪ Methanosarcina sp.
        ▪ Methanosarcina sp. FR
        ▪ Methanosarcina sp. GS1-A
        ▪ Methanosarcina sp. WH-1
□ Methanopyri
  □ Methanopyrales
    □ Methanopyraceae
      □ Methanopyrus
        ▪ Methanopyrus kandleri
□ Thermococci
  □ Thermococcales
    □ Thermococcaceae
      □ Palaeococcus
        ▪ Palaeococcus ferrophilus
      □ Pyrococcus
        ▪ Pyrococcus abyssi
        ▪ Pyrococcus endeavori
        □ Pyrococcus furiosus
          ▪ Pyrococcus furiosus DSM 3638
        ▪ Pyrococcus glycovorans
        ▪ Pyrococcus horikoshii
        ▪ Pyrococcus woesei
        ▪ Pyrococcus sp.
        ▪ Pyrococcus sp. GB-3A
        ▪ Pyrococcus sp. GB-D
        ▪ Pyrococcus sp. GE23
        ▪ Pyrococcus sp. GI-H
        ▪ Pyrococcus sp. GI-J
        ▪ Pyrococcus sp. JT1
        ▪ Pyrococcus sp. MZ14
        ▪ Pyrococcus sp. MZ4
        ▪ Pyrococcus sp. ST700
      □ Thermococcus
        ▪ Thermococcus acidaminovorans
        ▪ Thermococcus aegaeus
        ▪ Thermococcus aggregans
        ▪ Thermococcus alcaliphilus
        ▪ Thermococcus atlantis
        ▪ Thermococcus barophilus
        ▪ Thermococcus barossii
        ▪ Thermococcus celer
        ▪ Thermococcus chitonophagus
        ▪ Thermococcus fumicolans
        ▪ Thermococcus gammatolerans
        ▪ Thermococcus gorgonarius
        ▪ Thermococcus guaymasensis
        ▪ Thermococcus hydrothermalis
        ▪ Thermococcus kodakaraensis
        ▪ Thermococcus litoralis
        ▪ Thermococcus marinus
        ▪ Thermococcus mexicalis
        ▪ Thermococcus pacificus
        ▪ Thermococcus peptonophilus
        ▪ Thermococcus profundus
        ▪ Thermococcus radiophilus
        ▪ Thermococcus sibiricus
        ▪ Thermococcus siculi
        ▪ Thermococcus stetteri
        ▪ Thermococcus sulfurophilus
        ▪ Thermococcus waimanguensis
        ▪ Thermococcus waiotapuensis
        ▪ Thermococcus zilligii
        ▪ Thermococcus sp.
        ▪ Thermococcus sp. 9N2
        ▪ Thermococcus sp. 9N3
        ▪ Thermococcus sp. 9oN-7
        ▪ Thermococcus sp. B1001
        ▪ Thermococcus sp. CAR-80
        ▪ Thermococcus sp. CKU-1
        ▪ Thermococcus sp. CKU-199
        ▪ Thermococcus sp. CL1
        ▪ Thermococcus sp. CL2
        ▪ Thermococcus sp. CMI
        ▪ Thermococcus sp. CNR-5
        ▪ Thermococcus sp. CX1
        ▪ Thermococcus sp. CX2
        ▪ Thermococcus sp. CX3
        ▪ Thermococcus sp. CX4
        ▪ Thermococcus sp. CYA
        ▪ Thermococcus sp. GE8
        ▪ Thermococcus sp. Gorda2
        ▪ Thermococcus sp. Gorda3
        ▪ Thermococcus sp. Gorda4
        ▪ Thermococcus sp. Gorda5
        ▪ Thermococcus sp. Gorda6
        ▪ Thermococcus sp. JDF-3
        ▪ Thermococcus sp. KS-1
        ▪ Thermococcus sp. KS-8
        ▪ Thermococcus sp. MZ1
        ▪ Thermococcus sp. MZ10
        ▪ Thermococcus sp. MZ11
        ▪ Thermococcus sp. MZ12
        ▪ Thermococcus sp. MZ13
        ▪ Thermococcus sp. MZ2
        ▪ Thermococcus sp. MZ3
        ▪ Thermococcus sp. MZ5
        ▪ Thermococcus sp. MZ6
        ▪ Thermococcus sp. MZ8
        ▪ Thermococcus sp. MZ9
        ▪ Thermococcus sp. P6
        ▪ Thermococcus sp. Rt3
        ▪ Thermococcus sp. SN531
        ▪ Thermococcus sp. TK1
        ▪ Thermococcus sp. vp197
□ Thermoplasmata
  □ Thermoplasmatales
    □ Ferroplasmaceae
      □ Ferroplasma
        ▪ Ferroplasma acidarmanus
        ▪ Ferroplasma acidiphilum
    □ Picrophilaceae
      □ Picrophilus
        ▪ Picrophilus oshimae
        ▪ Picrophilus torridus
    □ Thermoplasmataceae
      □ Thermoplasma
        ▪ Thermoplasma acidophilum
        ▪ Thermoplasma volcanium
        ▪ Thermoplasma sp. XT101
        ▪ Thermoplasma sp. XT102
        ▪ Thermoplasma sp. XT103
        ▪ Thermoplasma sp. XT107
□ Korarchaeota
  ▪ korarchaeote SRI-306
```

Preparing Mutant Thermostable DNA Polymerase With Increased Reverse Transcriptase (RT) Activity

Cloned wild-type or mutant DNA polymerases may be modified to generate mutant forms exhibiting increased RT activity by a number of methods. These include the methods described below and other methods known in the art. Any thermostable DNA polymerase can be used to prepare the DNA polymerase mutants with increased RT activity in the invention.

A preferred method of preparing a DNA polymerase with increased RT activity is by genetic modification (e.g., by modifying the DNA sequence encoding a wild-type or mutant DNA polymerase). A number of methods are known in the art that permit the random as well as targeted mutation of DNA sequences (see for example, Ausubel et. al. Short Protocols in Molecular Biology (1995) 3^{rd} Ed. John Wiley & Sons, Inc.).

First, methods of random mutagenesis which will result in a panel of mutants bearing one or more randomly-situated mutations exist in the art. Such a panel of mutants may then be screened for those exhibiting increased RT activity relative to a wild-type polymerase (see “Methods of Evaluating Mutants for Increased RT Activity”, below). An example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase.

Second, there are a number of site-directed mutagenesis methods known in the art which allow one to mutate a particular site or region in a straightforward manner. There are a number of kits available commercially for the performance of site-directed mutagenesis, including both conventional and PCR-based methods. Useful examples include the EXSITE™ PCR-Based Site-directed Mutagenesis Kit available from Stratagene (Catalog No. 200502; PCR based) and the QUIKCHANGE™ Site-directed mutagenesis Kit from Stratagene (Catalog No. 200518; non-PCR-based), and the CHAMELEON® double-stranded Site-directed mutagenesis kit, also from Stratagene (Catalog No. 200509).

In addition DNA polymerases with increased RT activity may be generated by insertional mutation or truncation (N-terminal, internal or C-terminal) according to methodology known to a person skilled in the art.

Older methods of site-directed mutagenesis known in the art relied upon sub-cloning of the sequence to be mutated into a vector, such as an M13 bacteriophage vector, that allows the isolation of single-stranded DNA template. In these methods one annealed a mutagenic primer (i.e., a primer capable of annealing to the site to be mutated but bearing one or mismatched nucleotides at the site to be mutated) to the single-stranded template and then polymerized the complement of the template starting from the 3′ end of the mutagenic primer. The resulting duplexes were then transformed into host bacteria and plaques were screened for the desired mutation.

More recently, site-directed mutagenesis has employed PCR methodologies, which have the advantage of not requiring a single-stranded template. In addition, methods have been developed that do not require sub-cloning. Several issues may be considered when PCR-based site-directed mutagenesis is performed. First, in these methods it may be desirable to reduce the number of PCR cycles to prevent expansion of undesired mutations introduced by the polymerase. Second, a selection may be employed in order to reduce the number of non-mutated parental molecules persisting in the reaction. Third, an extended-length PCR method may be preferred in order to allow the use of a single PCR primer set. And fourth, because of the non-template-dependent terminal extension activity of some thermostable polymerases it may be necessary to incorporate an end-polishing step into the procedure prior to blunt-end ligation of the PCR-generated mutant product.

In some embodiments, a wild-type DNA polymerase is cloned by isolating genomic DNA or cDNA using molecular biological methods to serve as a template for mutagenesis. Alternatively, the genomic DNA or cDNA may be amplified by PCR and the PCR product may be used as template for mutagenesis.

The unlimiting protocol described below accommodates these considerations through the following steps. First, the template concentration used is approximately 1000-fold higher than that used in conventional PCR reactions, allowing a reduction in the number of cycles from 25-30 down to 5-10 without dramatically reducing product yield. Second, the restriction endonuclease DpnI (recognition target sequence: 5-Gm6ATC-3, where the A residue is methylated) is used to select against parental DNA, since most common strains of E. coli Dam methylate their DNA at the sequence 5-GATC-3. Third, Taq Extender is used in the PCR mix in order to increase the proportion of long (i.e., full plasmid length) PCR products. Finally, Pfu DNA polymerase is used to polish the ends of the PCR product prior to intramolecular ligation using T4 DNA ligase.

One method is described in detail as follows for PCR-based site directed mutagenesis according to one embodiment of the invention.

Plasmid template DNA comprising a DNA polymerase encoding polynucleotide (approximately 0.5 pmole) is added to a PCR cocktail containing: 1× mutagenesis buffer (20 mM Tris HCl, pH 7.5; 8 mM MgCl₂; 40 μg/ml BSA); 12-20 pmole of each primer (one of skill in the art may design a mutagenic primer as necessary, giving consideration to those factors such as base composition, primer length and intended buffer salt concentrations that affect the annealing characteristics of oligonucleotide primers; one primer must contain the desired mutation within the DNA polymerase encoding sequence, and one (the same or the other) must contain a 5′ phosphate to facilitate later ligation), 250 uM each dNTP, 2.5 U Taq DNA polymerase, and 2.5 U of Taq Extender (Available from Stratagene; See Nielson et al. (1994) Strategies 7: 27, and U.S. Pat. No. 5,556,772).

Primers can be prepared using the triester method of Matteucci et al., 1981, J. Am. Chem. Soc. 103:3185-3191, incorporated herein by reference. Alternatively automated synthesis may be preferred, for example, on a Biosearch 8700 DNA Synthesizer using cyanoethyl phosphoramidite chemistry.

The PCR cycling is performed as follows: 1 cycle of 4 min at 94° C., 2 min at 50° C. and 2 min at 72° C.; followed by 5-10 cycles of 1 min at 94° C., 2 min at 54° C. and 1 min at 72° C. The parental template DNA and the linear, PCR-generated DNA incorporating the mutagenic primer are treated with DpnI (10 U) and Pfu DNA polymerase (2.5U). This results in the DpnI digestion of the in vivo methylated parental template and hybrid DNA and the removal, by Pfu DNA polymerase, of the non-template-directed Taq DNA polymerase-extended base(s) on the linear PCR product. The reaction is incubated at 37° C. for 30 min and then transferred to 72° C. for an additional 30 min. Mutagenesis buffer (115 μl of 1×) containing 0.5 mM ATP is added to the DpnI-digested, Pfu DNA polymerase-polished PCR products. The solution is mixed and 10 μl are removed to a new microfuge tube and T4 DNA ligase (2-4 U) is added. The ligation is incubated for greater than 60 min at 37° C. Finally, the treated solution is transformed into competent E. coli according to standard methods.

Direct comparison of Family B DNA polymerases from diverse organisms, including thermostable Family B DNA polymerases indicates that the domain structure of these enzymes is highly conserved (See, e.g., Hopfner et al., 1999, Proc. Natl. Acad Sci. U.S.A. 96: 3600-3605; Blanco et al., 1991, Gene 100: 27-38; and Larder et al., 1987, EMBO J. 6: 169-175). All Family B DNA polymerases have six conserved regions, designated Regions I-VI, and arranged in the polypeptides in the order IV-II-VI-III-I-V (separation between the Regions varies, but the order does not). Region I (also known as Motif C) is defined by the conserved sequence D T D, located at amino acids 541-543 in Pfu DNA polymerase and at amino acids 540-542 in JDF-3 DNA polymerase. Region II (also known as Motif A) is defined by the consensus sequence D X X (A/S) L Y P S I, locatred at amino acids 405-413 in Pfu DNA polymerase and at amino acids 404-412 in JDF-3 DNA polymerase. Region III (also known as Motif B) is defined by the consensus sequence K X X X N A/S X Y G, located at amino acids 488-496 in Pfu DNA polymerase and at amino acids 487-495 in JDF-3 DNA polymerase. Sequence alignments of these sequences with those of other Family B DNA polymerases permit the assignment of the boundaries of the various Regions on other Family B DNA polymerases. The crystal structures have been solved for several Family B DNA polymerases, including Thermococcus gorgonarius (Hopfner et al., 1999, Proc. Natl. Acad. Sci. U.S.A. 96: 3600-3605), 9° N (Rodrigues et al., 2000, J. Mol. Biol. 299: 447-462), and Thermococcus sp. strain KODI (formerly classified as a Pyrococcus sp., Hashimoto et al., 2001, J. Mol. Biol. 306: 469-477), aiding in the establishment of structure/function relationships for the Regions. The location of these conserved regions provides a useful model to direct genetic modifications for preparing DNA polymerase with increased RT activity whilst conserving essential functions e.g. DNA polymerization and proofreading activity. For example, it is recognized herein that the “LYP” structural motif that is part of the larger conserved structural motif DXXSLYPSI defining Region II is a primary target for mutations that enhance the reverse transcriptase activity of the enzyme. As used herein, the term “LYP motif” means an amino acid sequence within Region II of a Family B DNA polymerase that corresponds in a sequence alignment, performed using BLAST or Clustal W, to the LYP sequence located at amino acids 408 to 410 of the JDF-3 Family B DNA polymerase of SEQ ID NO: 1 (the LYP motif of Pfu DNA polymerase is located at amino acids 409-411 of the polypeptide). It is noted that while the motif is most frequently LYP, there are members of the Archaeal Family B DNA polymerases that vary in this motif—for example, the LYP corresponds to MYP in Archaeoglobus fulgidusfu (Afu) DNA polymerase.

As disclosed herein, amino acid changes at the position corresponding to L408 of SEQ ID NO: 1 which lead to increased reverse transcriptase activity tend to introduce cyclic side chains, such as phenylalanine, tryptophan, histidine or tyrosine. While the amino acids with cyclic side chains are demonstrated herein to increase the reverse transcriptase activity of Archaeal Family B DNA polymerases, other amino acid changes at the LYP motif are contemplated to have effects on the reverse transcriptase activity. Thus, in order to modify the reverse transcriptase activity of another Archaeal Family B DNA polymerase, one would first look to modify the LYP motif of Region II, particularly the L or other corresponding amino acid of the LYP motif, first substituting cyclic side chains and assessing reverse transcriptase activity relative to wild-type as disclosed herein below in “Methods of Evaluating Mutants for Increased RT Activity.” If necessary or if desired, one can subsequently modify the same position in the LYP motif with additional amino acids and similarly assess the effect on activity. Alternatively, or in addition, one can modify the other positions in the LYP motif and similarly assess the reverse transcriptase activity.

A degenerate oligonucleotide primer may be used for generating DNA polymerase mutants of the present invention. In some embodiments, chemical synthesis of a degenerate primer is carried out in an automatic DNA synthesizer, and the purpose of a degenerate primer is to provide, in one mixture, all of the sequences encoding a specific desired mutation site of the DNA polymerase sequence. The synthesis of degenerate oligonucleotides is well known in the art (e.g., Narang, S. A, Tetrahedron 39:3 9, 1983; Itakura et al., Recombinant DNA, Proc 3rd Cleveland Sympos. Macromol., Walton, ed., Elsevier, Amsterdam, pp 273-289, 1981; Itakura et al., Annu. Rev. Biochem. 53:323, 1984; Itakura et al., Science 198:1056, 1984; and Ike et al., Nucleic Acid Res. 11:477 1983). Such techniques have been employed in the directed evolution of other proteins (e.g., Scott et al., Science 249:386-390, 1980; Roberts et al., Proc. Nat'l. Acad. Sci., 89:2429-2433, 1992; Devlin et al., Science 249: 404-406, 1990; Cwirla et al., Proc. Nat'l. Acad. Sci., 87: 6378-6382, 1990; as well as U.S. Pat. Nos. 5,223,409, 5,198,346, and 5,096,815, each of which is incorporated herein by reference).

A polynucleotide encoding a mutant DNA polymerase with increased RT activity may be screened and/or confirmed by methods known in the art, such as described below in Methods of Evaluating Mutants for Increased RT Activity.

Polynucleotides encoding the desired mutant DNA polymerases generated by mutagenesis may be sequenced to identify the mutations. For those mutants comprising more than one mutation, the effect of a given mutation may be evaluated by introduction of the identified mutation to the wild-type gene by site-directed mutagenesis in isolation from the other mutations borne by the particular mutant. Screening assays of the single mutant thus produced will then allow the determination of the effect of that mutation alone.

In a preferred embodiment, the enzyme with increased RT activity is derived from an Archaeal DNA polymerase containing one or more mutations.

In a preferred embodiment, the enzyme with increased RT activity is derived from a Pfu or JDF-3 DNA polymerase.

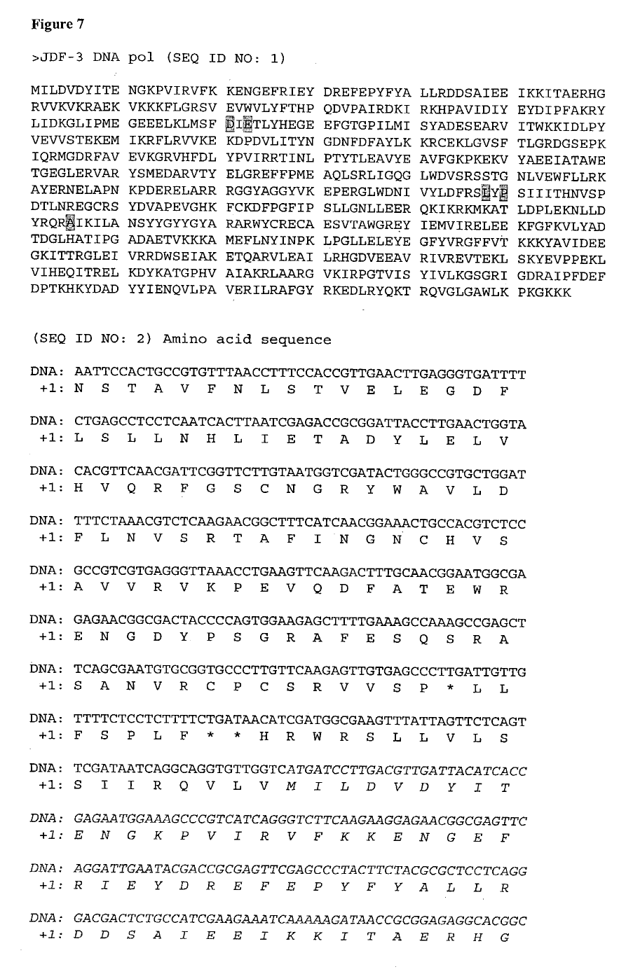

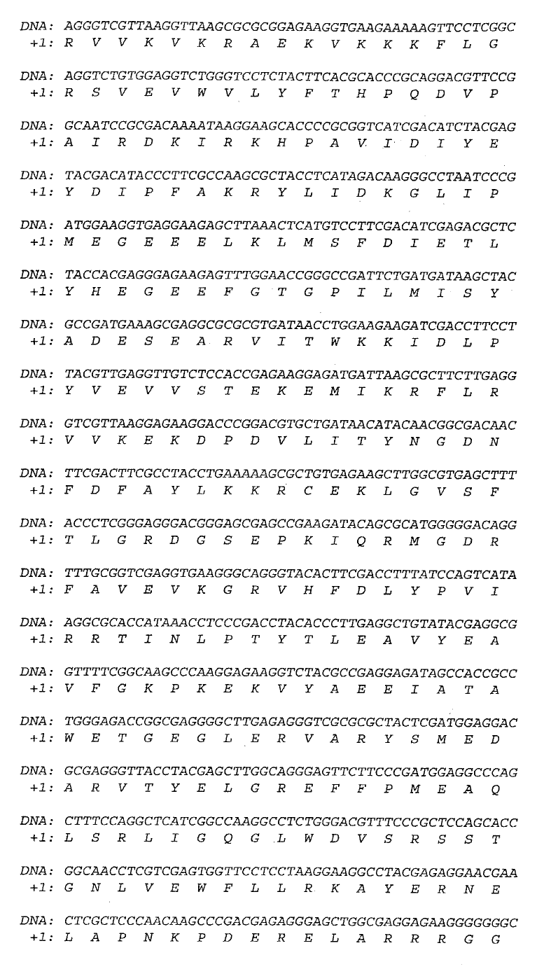

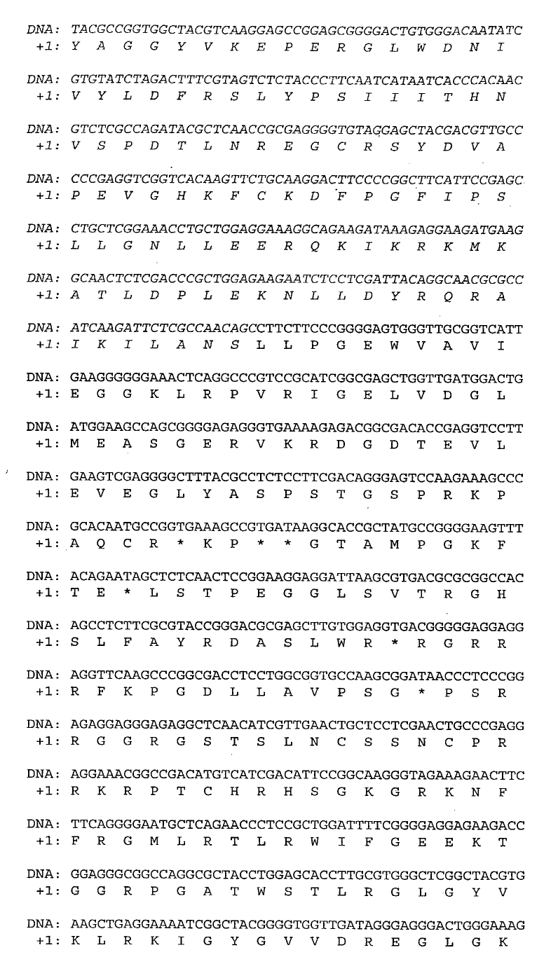

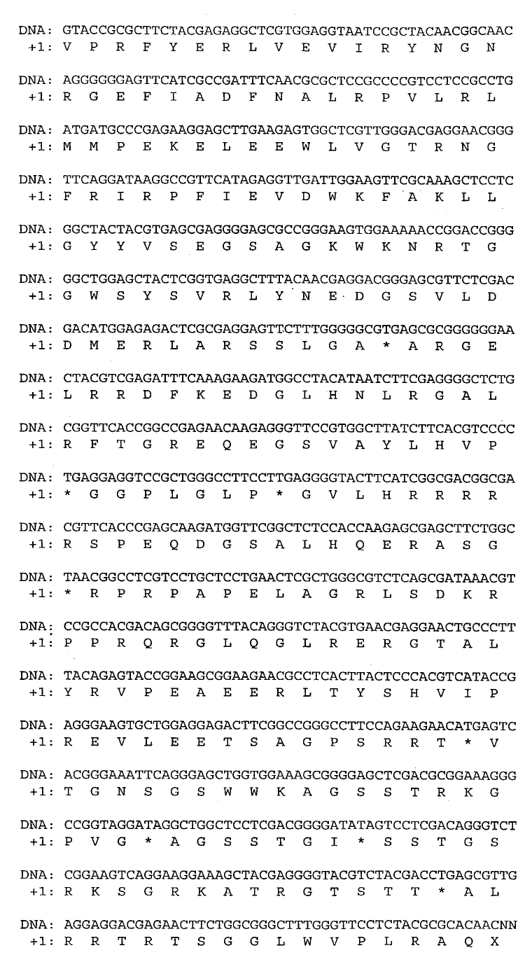

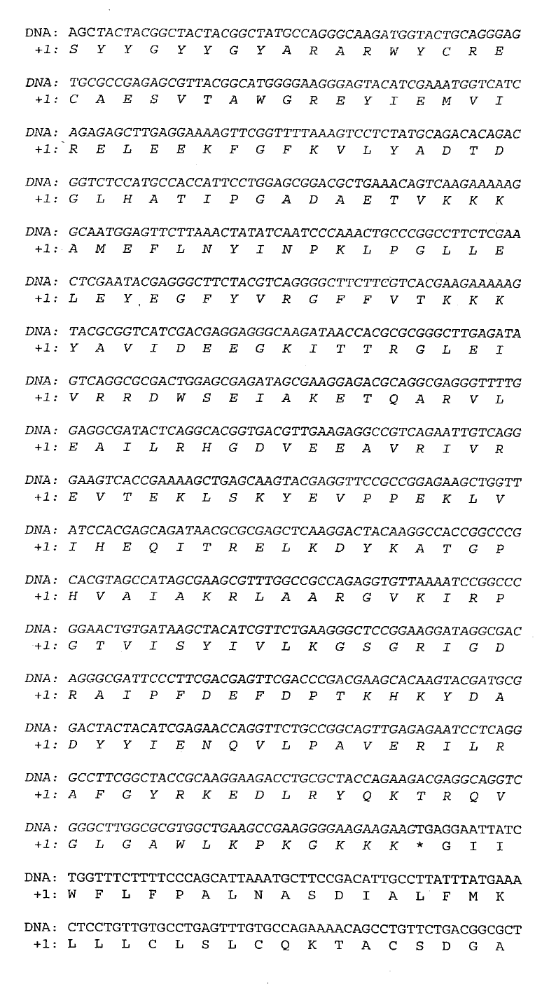

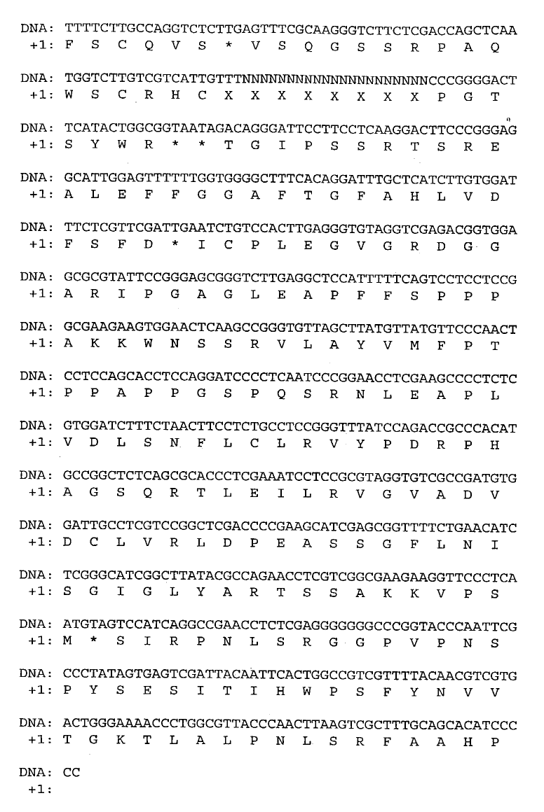

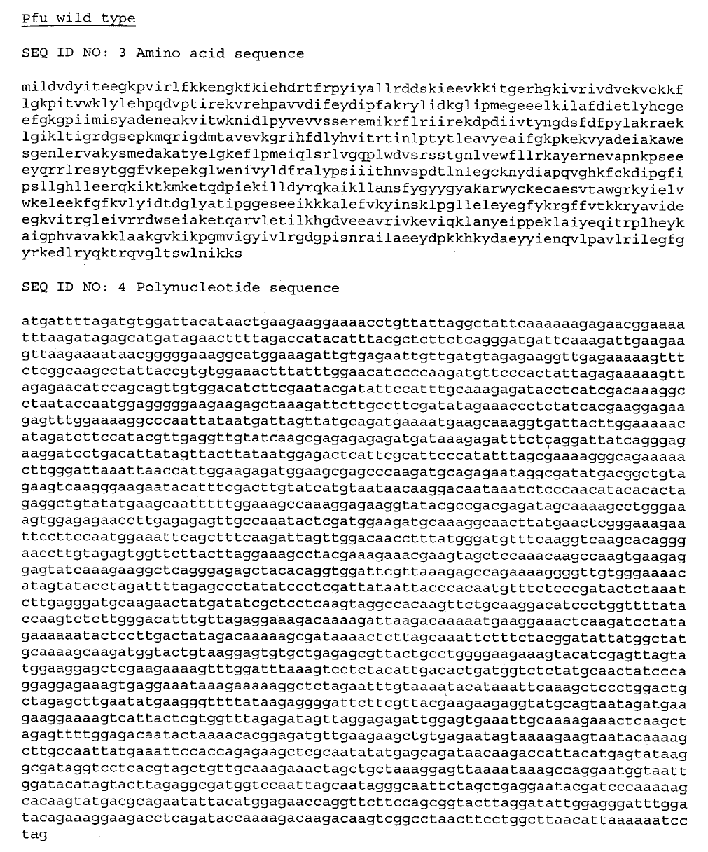

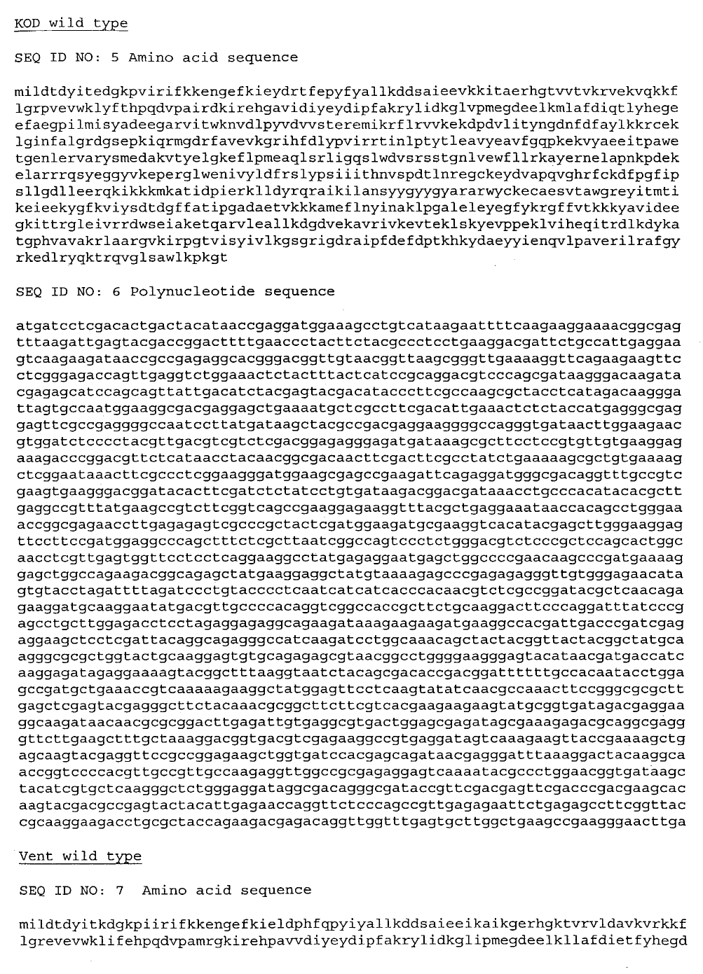

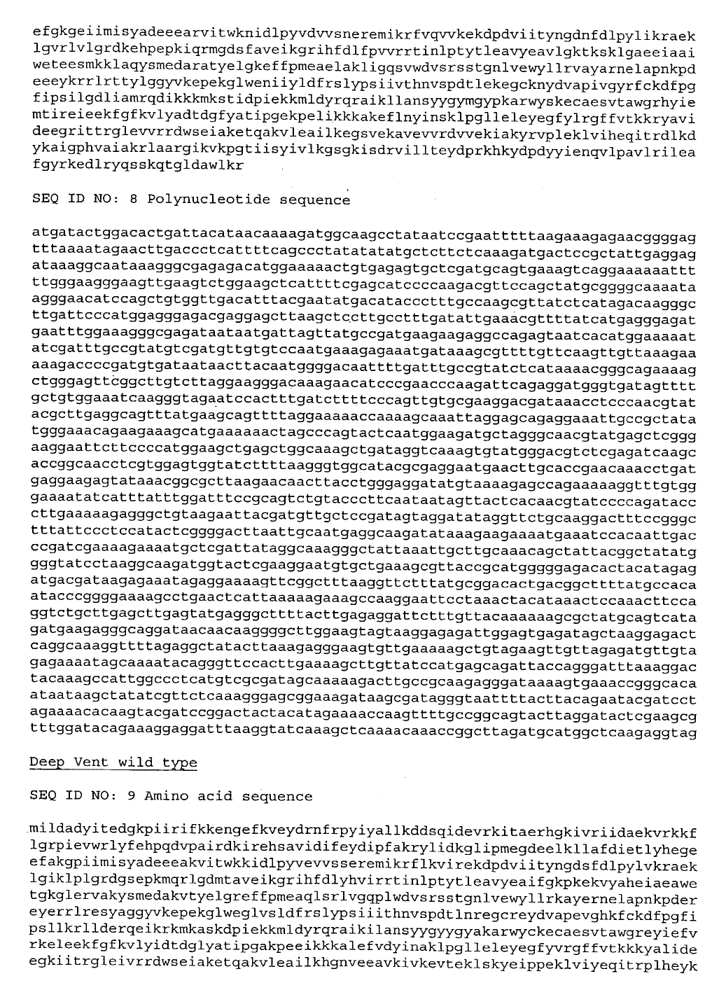

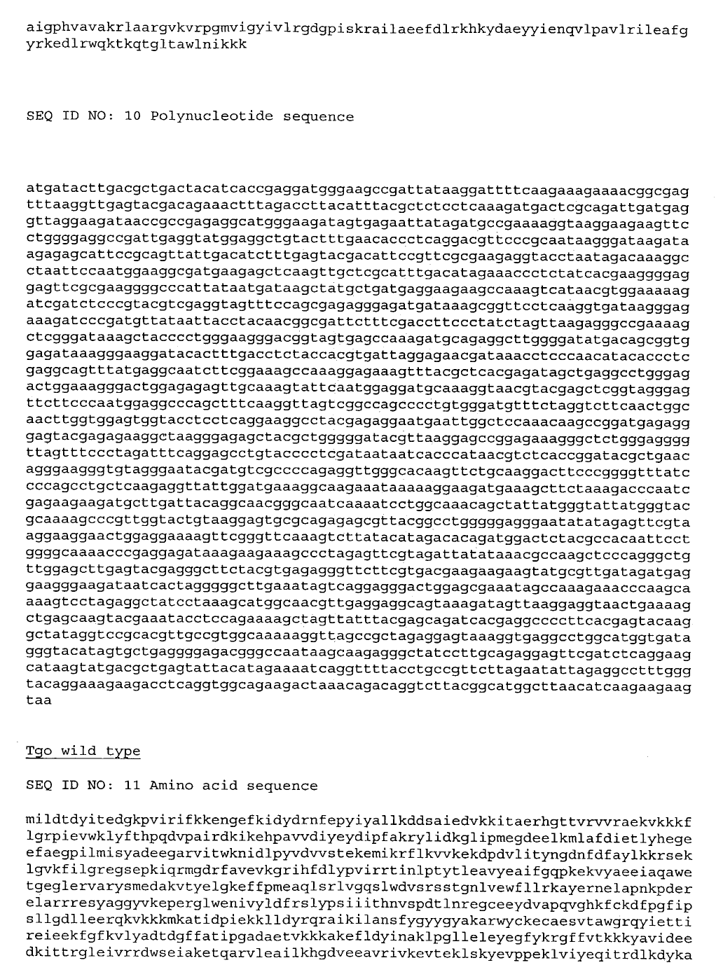

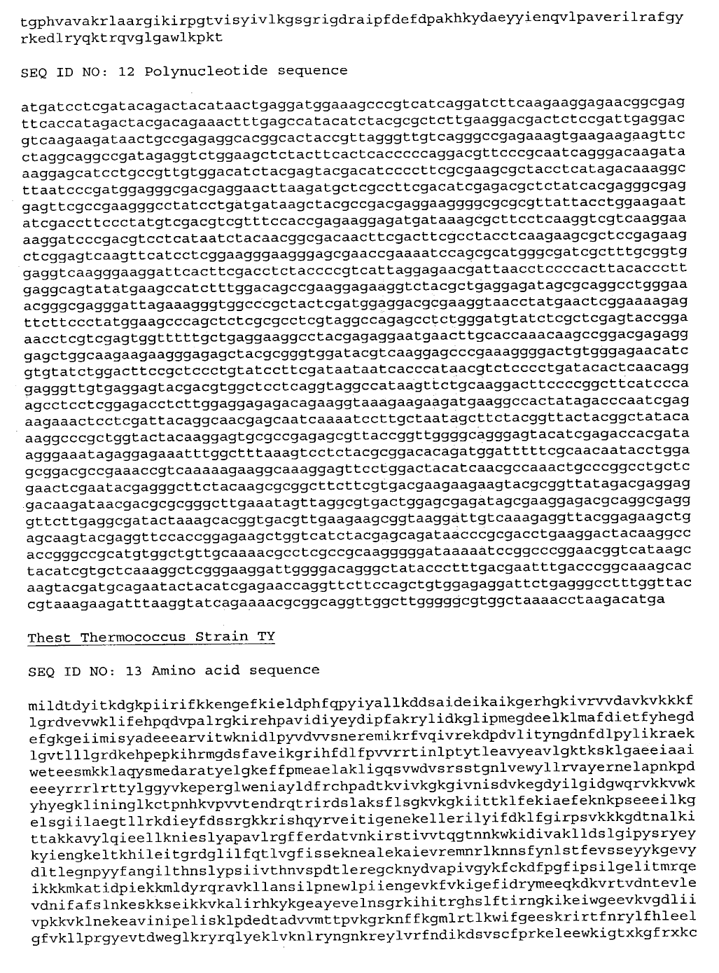

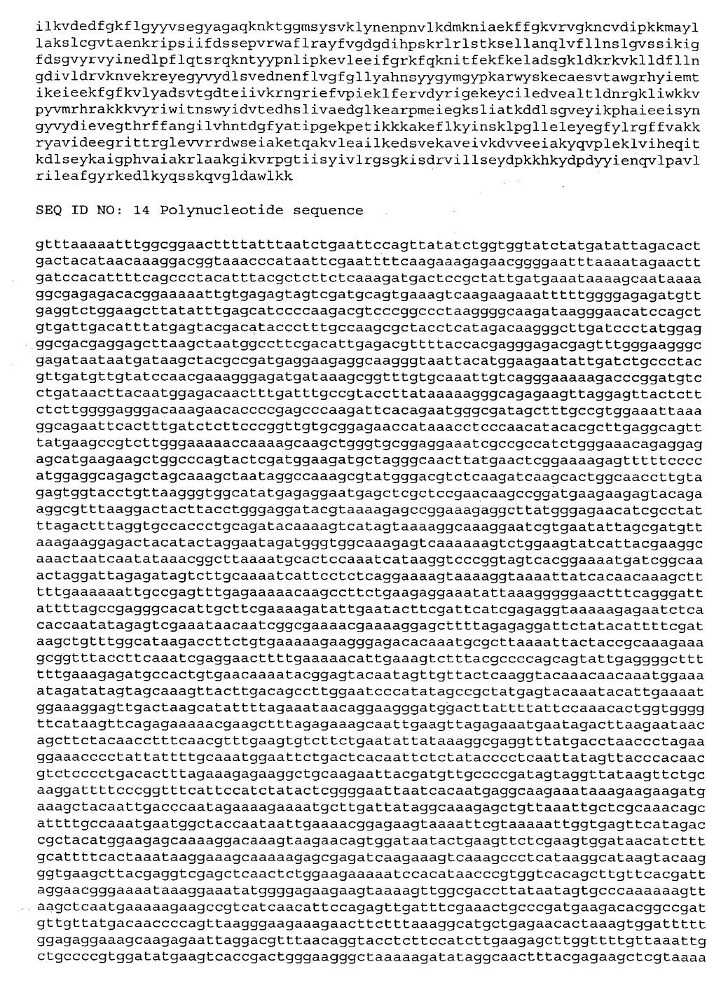

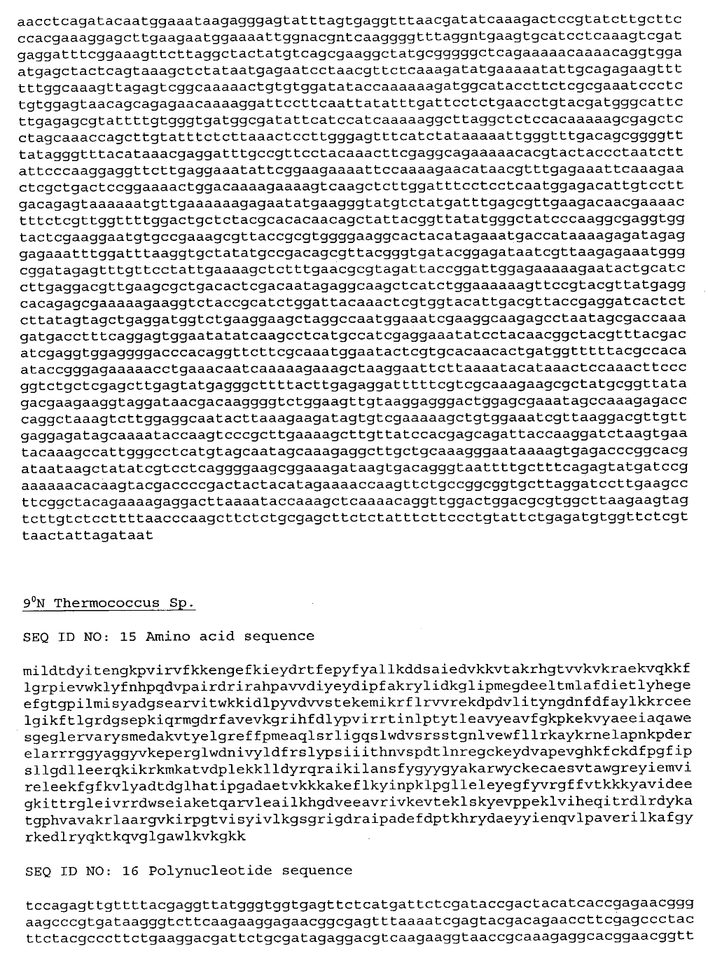

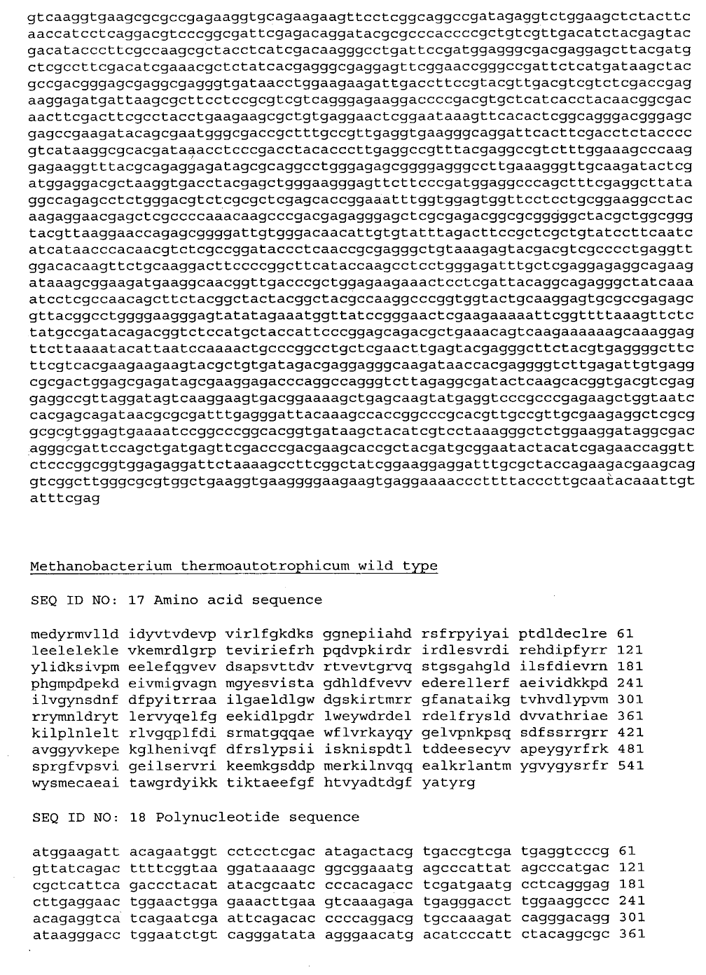

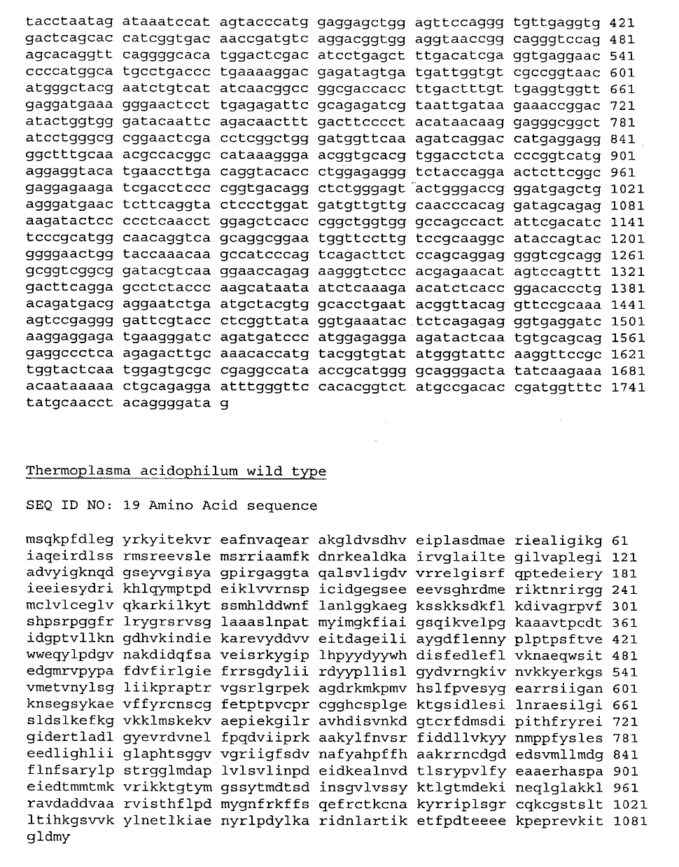

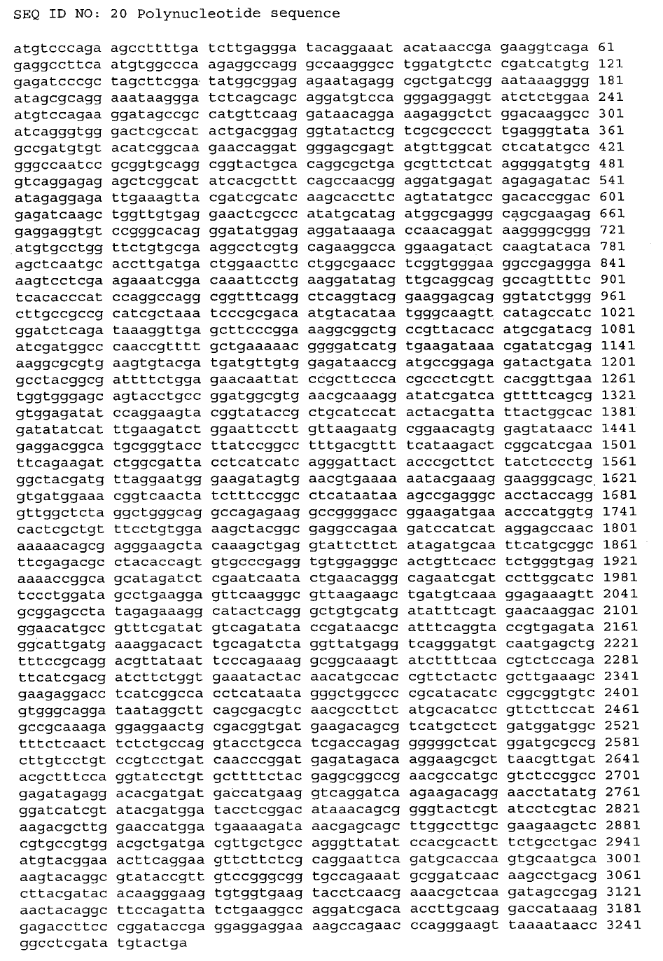

The amino acid and DNA coding sequence of a wild-type Pfu or JDF-3 DNA polymerase are shown in FIG. 7 (Genbank Accession # P80061 (PFU) and Q56366 (JDF-3), respectively). A detailed description of the structure and function of Pfu DNA polymerase can be found, among other places, in U.S. Pat. Nos. 5,948,663; 5,866,395; 5,545,552; 5,556,772, while a detailed description of the structure and function of JDF-3 DNA polymerase can be found, among other places, in U.S. Pat. Nos. 5,948,663; 5,866,395; 5,545,552; 5,556,772, all of which are hereby incorporated by reference. A non-limiting detailed procedure for preparing Pfu or a JDF-3 DNA polymerase with increased RT activity is provided in the Examples herein.

A person of ordinary skill in the art having the benefit of this disclosure will recognize that polymerases with reduced uracil detection activity derived from Archaeal DNA polymerases, including Vent DNA polymerase, JDF-3 DNA polymerase, Pfu polymerase, Tgo DNA polymerase, KOD, other enzymes listed in Tables II and III, and the like may be suitably used in the present invention.

The enzyme of the subject composition may comprise DNA polymerases that have not yet been isolated.

In preferred embodiments of the invention, the mutant Archaeal DNA polymerase harbors an amino acid substitution at amino acid position corresponding to L409 of the Pfu DNA polymerase (see FIG. 6). In a preferred embodiment, the mutant DNA polymerase of the invention contains a Leucine to F, Y, W or H substitution at the amino acid at a position corresponding to L408 of the JDF-3 Polymerase or L409 of the Pfu DNA polymerase.

In one embodiment, the mutant DNA polymerase of the present invention is a Pfu DNA polymerase that contains a Leucine to F, Y, W or H substitution at amino acid position 409.

In one embodiment, the mutant DNA polymerase of the present invention is a JDF-3 DNA polymerase that contains a Leucine to F, Y, W or H substitution at amino acid position 408.

According to the invention, LYP motif mutant DNA polymerases (e.g., Pfu L409 mutant or JDF-3 L408 mutant) with increased RT activity may contain one or more additional mutations that further increases its RT activity, or reduce or abolish one or more additional activities of the DNA polymerases, e.g., 3′-5′ exonuclease activity.

In one embodiment, an L409 mutant Pfu DNA polymerase according to the invention contains one or more additional mutations that result in a form which is substantially lacking 3′-5′ exonuclease activity.

The invention further provides for L409 mutant Pfu DNA polymerases with increased RT activity further containing one or mutations that reduce or eliminate 3′-5′ exonuclease activity as disclosed in the pending U.S. patent application Ser. No. 09/698,341 (Sorge et al; filed Oct. 27, 2000).

In a preferred embodiment, the invention provides for a L409/D141/E143 triple mutant Pfu DNA polymerase with reduced 3′-5′ exonuclease activity and increased RT activity.

In one embodiment, the triple mutant Pfu DNA polymerase contains an F, Y, W or H substitution at L409, an A substitution at D141, and an A substitution at E143.

DNA polymerases containing multiple mutations may be generated by site directed mutagenesis using a polynucleotide encoding a DNA polymerase mutant already possessing a desired mutation, or they may be generated by using one or more mutagenic primers containing one or more according to methods that are well known in the art and are described herein.

Methods used to generate 3′-5′ exonuclease deficient JDF-3 DNA polymerases including the D141A and E143A mutations are disclosed in the pending U.S. patent application Ser. No. 09/698,341 (Sorge et al; filed Oct. 27, 2000). A person skilled in the art in possession of the L409 Pfu DNA polymerase cDNA and the teachings of the pending U.S. patent application Ser. No. 09/698,341 (Sorge et al; filed Oct. 27, 2000) would have no difficulty introducing both the corresponding D141A and E143A mutations or other 3′-5′ exonuclease mutations into the L409 Pfu DNA polymerase cDNA, as disclosed in the pending U.S. patent application Ser. No. 09/698,341, using established site directed mutagenesis methodology.

In another embodiment, a mutant archaeal DNA polymerase is a chimeric protein, for example, further comprising a domain that increases processivity and/or increases salt resistance. A domain useful according to the invention and methods of preparing chimeras are described in WO 01/92501 A1 and Pavlov et al., 2002, Proc. Natl. Acad. Sci USA, 99:13510-13515. Both references are herein incorporated in their entirety.

In light of the present disclosure, other forms of mutagenesis generally applicable will be apparent to those skilled in the art in addition to the aforementioned mutagenesis methods. For example, DNA polymerase mutants can be generated and screened using, for example, alanine scanning mutagenesis and the like (Ruf et al., Biochem., 33:1565-1572, 1994; Wang et al., J. Biol. Chem., 269:3095-3099, 1994; Balint et al. Gene 137:109-118, 1993; Grodberg et al., Eur. J. Biochem., 218:597-601, 1993; Nagashima et al., J. Biol. Chem., 268:2888-2892, 1993; Lowman et al., Biochem., 30:10832-10838, 1991; and Cunningham et al., Science, 244:1081-1085, 1989); linker scanning mutagenesis (Gustin et al., Virol., 193:653-660, 1993; Brown et al., Mol. Cell. Biol., 12:2644-2652, 1992; McKnight et al., Science, 232:316); or saturation mutagenesis (Meyers et al., Science, 232:613, 1986), all references hereby incorporated by reference.

Methods of Evaluating Mutants for Increased RT Activity.

A wide range of techniques are known in the art for screening polynucleotide products of mutagenesis. The most widely used techniques for screening large number of polynucleotide products typically comprise cloning the mutagenesis polynucleotides into replicable expression vectors, transforming appropriate cells with the resulting vectors, and expressing the polynucleotides under conditions such that detection of a desired activity (e.g., RT) facilitates relatively easy isolation of the vector containing the polynucleotide encoding the desired product.

Methods for assaying reverse transcriptase (RT) activity based on the RNA-dependent synthesis of DNA have been well known in the art, e.g., as described in U.S. Pat. No. 3,755,086; Poiesz et al., (1980) Proc. Natl. Acad. Sci. USA, 77: 1415; Hoffman et al., (1985) Virology 147: 326; all hereby incorporated by reference.

Recently, highly sensitive PCR based assays have been developed that can detect RNA-dependent DNA polymerase in the equivalent of one to ten particles (Silver et al. (1993) Nucleic Acids Res. 21: 3593-4; U.S. Pat. No. 5,807,669). One such assay, designated as PBRT (PCR-based reverse transcriptase), has been used to detect RT activity in a variety of samples (Pyra et al. (1994) Proc. Natl. Acad. Sci. USA 51: 1544-8; Boni, et al. (1996) J. Med. Virol. 49: 23-28). This assay is 106-107 more sensitive than the conventional RT assay.

Other useful RT assays include, but are not limited to, one-step fluorescent probe product-enhanced reverse transcriptase assay described in Hepler, R. W., and Keller, P. M. (1998). Biotechniques 25(1), 98-106; an improved product enhanced reverse transcriptase assay described in Chang, A., Ostrove, J. M., and Bird, R. E. (1997) J Virol Methods 65(1), 45-54; an improved non-radioisotopic reverse transcriptase assay described in Nakano et al., (1994) Kansenshogaku Zasshi 68(7), 923-3 1; a highly sensitive qualitative and quantitative detection of reverse transcriptase activity as described in Yamamoto, S., Folks, T. M., and Heneine, W. (1996) J Virol Methods 61(1-2), 135-43, all references hereby incorporated by reference.

RT activity can be measured using radioactive or non-radioactive labels.

In one embodiment, 1 μl of appropriately purified DNA polymerase mutant or diluted bacterial extract (i.e., heat-treated and clarified extract of bacterial cells expressing a cloned polymerase or mutated cloned polymerase) is added to 10 μl of each nucleotide cocktail (200 μM dATP, 200 μM dGTP, 200 μM dCTP and 5 μCi/ml α-³³P dCTP and 200 μM dTTP, a RNA template, 1× appropriate buffer, followed by incubation at the optimal temperature for 30 minutes (e.g., 72° C. for Pfu DNA polymerase), for example, as described in Hogrefe et al., 2001, Methods in Enzymology, 343:91-116. Extension reactions are then quenched on ice, and 5 μl aliquots are spotted immediately onto DE81 ion-exchange filters (2.3 cm; Whatman #3658323). Unincorporated label is removed by 6 washes with 2×SCC (0.3M NaCl, 30 mM sodium citrate, pH 7.0), followed by a brief wash with 100% ethanol. Incorporated radioactivity is then measured by scintillation counting. Reactions that lack enzyme are also set up along with sample incubations to determine “total cpms” (omit filter wash steps) and “minimum cpms” (wash filters as above). Cpms bound is proportional to the amount of RT activity present per volume of bacterial extract or purified DNA polymerase.

In another embodiment, the RT activity is measured by incorporation of non-radioactive digoxigenin labeled dUTP into the synthesized DNA and detection and quantification of the incorporated label essentially according to the method described in Holtke, H.-J.; Sagner, G; Kessler, C. and Schmitz, G. (1992) Biotechniques 12, 104-113. The reaction is performed in a reaction mixture consists of the following components: 1 μg of polydA-(dT)₁₅, 33 μM of dTTP, 0.36 μM of labeled-dUTP, 200 mg/ml BSA, 10 mM Tris-HCl, pH 8.5, 20 mM KCl, 5 mM MgCl₂, 10 mM DTE and various amounts of DNA polymerase. The samples are incubated for 30 min. at 50° C., the reaction is stopped by addition of 2μ 0.5 M EDTA, and the tubes placed on ice. After addition of 8 μl 5 M NaCl and 150 μl of Ethanol (precooled to −20° C.) the DNA is precipitated by incubation for 15 min on ice and pelleted by centrifugation for 10 min at 13000×rpm and 4° C. The pellet is washed with 100 μl of 70% Ethanol (precooled to −20° C.) and 0.2 M NaCl, centrifuged again and dried under vacuum.

The pellets are dissolved in 50 μl Tris-EDTA (10 mM/0.1 mM; pH 7.5). 5 μl of the sample are spotted into a well of a nylon membrane bottomed white microwave plate (Pall Filtrationstechnik GmbH, Dreieich, FRG, product no: SM045BWP). The DNA is fixed to the membrane by baking for 10 min. at 70° C. The DNA loaded wells are filled with 100 μl of 0.45 μm-filtrated 1% blocking solution (100 mM maleic acid, 150 mM NaCl, 1% (w/v) casein, pH 7.5). All following incubation steps are done at room temperature. After incubation for 2 min. the solution is sucked through the membrane with a suitable vacuum manifold at −0.4 bar. After repeating the washing step, the wells are filled with 100 μl of a 1:10,000-dilution of Anti-digoxigenin-AP, Fab fragments (Boehringer Mannheim, FRG, no: 1093274) diluted in the above blocking solution. After incubation for 2 min. and sucking this step is repeated once. The wells are washed twice under vacuum with 200 μl each time washing-buffer 1 (100 mM maleic-acid, 150 mM NaCl, 0.3%(v/v) Tween.TM. 20, pH 7.5). After washing another two times under vacuum with 200 μl each time washing-buffer 2 (10 mM Tris-HCl, 100 mM NaCl, 50 mM MgCl₂, pH 9.5) the wells are incubated for 5 min with 50 μl of CSPD (Boehringer Mannheim, no: 1655884), diluted 1:100 in washing-buffer 2, which serves as a chemiluminescent substrate for the alkaline phosphatase. The solution is sucked through the membrane and after 10 min incubation the RLU/s (Relative Light Unit per second) are detected in a Luminometer e.g. MicroLumat LB 96 P (EG&G Berthold, Wilbad, FRG). With a serial dilution of Taq DNA polymerase a reference curve is prepared from which the linear range serves as a standard for the activity determination of the DNA polymerase to be analyzed.

U.S. Pat. No. 6,100,039 (incorporated hereby by reference) describes another useful process for detecting reverse transcriptase activity using fluorescence polarization: the reverse transcriptase activity detection assays are performed using a Beacon™ 2000 Analyzer. The following reagents are purchased from commercial sources: fluorescein-labeled oligo dA-F (Bio.Synthesis Corp., Lewisville, Tex.), AMV Reverse Transcriptase (Promega Corp., Madison, Wis.), and Polyadenylic Acid Poly A (Pharmacia Biotech, Milwaukee, Wis.). The assay requires a reverse trancriptase reaction step followed by a fluorescence polarization-based detection step. The reverse transcriptase reactions are completed using the directions accompanying the kit. In the reaction 20 ng of Oligo (dT) were annealed to 1 μg of Poly A at 70° C. for 5 minutes. The annealed reactions are added to an RT mix containing RT buffer and dTTP nucleotides with varying units of reverse transcriptase (30, 15, 7.5, 3.8, and 1.9 Units/Rxn). Reactions are incubated at 37° C. in a water bath. 5 μl aliquots are quenched at 5, 10, 15, 20, 25, 30, 45, and 60 minutes by adding the aliquots to a tube containing 20 μl of 125 mM NaOH. For the detection step, a 75 μl aliquot of oligo dA-F in 0.5 M Tris, pH 7.5, is added to each quenched reaction. The samples are incubated for 10 minutes at room temperature. Fluorescence polarization in each sample was measured using the Beacon™ 2000 Analyzer.

Expression of Wild-Type or Mutant Enzymes According to the Invention

Methods known in the art may be applied to express and isolate the mutated forms of DNA polymerase according to the invention. The methods described here can be also applied for the expression of wild-type enzymes useful in the invention. Many bacterial expression vectors contain sequence elements or combinations of sequence elements allowing high level inducible expression of the protein encoded by a foreign sequence. For example, as mentioned above, bacteria expressing an integrated inducible form of the T7 RNA polymerase gene may be transformed with an expression vector bearing a mutated DNA polymerase gene linked to the T7 promoter. Induction of the T7 RNA polymerase by addition of an appropriate inducer, for example, isopropyl-p-D-thiogalactopyranoside (IPTG) for a lac-inducible promoter, induces the high level expression of the mutated gene from the T7 promoter.

Appropriate host strains of bacteria may be selected from those available in the art by one of skill in the art. As a non-limiting example, E. coli strain BL-21 is commonly used for expression of exogenous proteins since it is protease deficient relative to other strains of E. coli. BL-21 strains bearing an inducible T7 RNA polymerase gene include WJ56 and ER2566 (Gardner & Jack, 1999, supra). For situations in which codon usage for the particular polymerase gene differs from that normally seen in E. coli genes, there are strains of BL-21 that are modified to carry tRNA genes encoding tRNAs with rarer anticodons (for example, argU, ileY, leuW, and proL tRNA genes), allowing high efficiency expression of cloned protein genes, for example, cloned archaeal enzyme genes (several BL21-CODON PLUSTM cell strains carrying rare-codon tRNAs are available from Stratagene, for example).

There are many methods known to those of skill in the art that are suitable for the purification of a mutant DNA polymerase of the invention. For example, the method of Lawyer et al. (1993, PCR Meth. & App. 2: 275) is well suited for the isolation of DNA polymerases expressed in E. coli, as it was designed originally for the isolation of Taq polymerase. Alternatively, the method of Kong et al. (1993, J. Biol. Chem. 268: 1965, incorporated herein by reference) may be used, which employs a heat denaturation step to destroy host proteins, and two column purification steps (over DEAE-Sepharose and heparin-Sepharose columns) to isolate highly active and approximately 80% pure DNA polymerase. Further, DNA polymerase mutants may be isolated by an ammonium sulfate fractionation, followed by Q Sepharose and DNA cellulose columns, or by adsorption of contaminants on a HiTrap Q column, followed by gradient elution from a HiTrap heparin column.

In one embodiment, the Pfu mutants are expressed and purified as described in U.S. Pat. No. 5,489,523, hereby incorporated by reference in its entirety.

In another embodiment, the JDF-3 mutants are expressed and purified as described in U.S. patent application Ser. No. 09/896,923, hereby incorporated by reference in its entirety.

Kits

The invention herein also contemplates a kit format which comprises a package unit having one or more containers of the subject composition and in some embodiments including containers of various reagents used for polynucleotide synthesis, including RT or RT-PCR.

It is contemplated that the kits of the present invention find use for methods including, but not limited to, reverse transcribing template RNA for the construction of cDNA libraries, for the reverse transcription of RNA for differential display PCR, and RT-PCR identification of target RNA in a sample suspected of containing the target RNA. In some embodiments, the RT or RT-PCR kit comprises the essential reagents required for the method of reverse transcription. For example, in some embodiments, the kit includes a vessel containing a polymerase with increased RT activity. In some embodiments, the concentration of polymerase ranges from about 0.1 to 100 u/μl; in other embodiments, the concentration is about 5 u/μl. In some embodiments, kits for reverse transcription also include a vessel containing a RT reaction buffer. Preferably, these reagents are free of contaminating RNase activity. In other embodiments of the present invention, reaction buffers comprise a buffering reagent in a concentration of about 5 to 15 mM (preferably about 10 mM Tris-HCl at a pH of about 7.5 to 9.0 at 25° C.), a monovalent salt in a concentration of about 20 to 100 mM (preferably about 50 mM NaCl or KCI), a divalent cation in a concentration of about 1.0 to 10.0 mM (preferably MgCl₂), dNTPs in a concentration of about 0.05 to 1.0 mM each (preferably about 0.2 mM each), and a surfactant in a concentration of about 0.001 to 1.0% by volume (preferably about 0.01% to 0.1%). In some embodiments, a purified RNA standard set is provided in order to allow quality control and for comparison to experimental samples. In some embodiments, the kit is packaged in a single enclosure including instructions for performing the assay methods (e.g., reverse transcription or RT-PCR). In some embodiments, the reagents are provided in containers and are of a strength suitable for direct use or use after dilution.

The composition or kit of the present invention may further comprise compounds for improving product yield, processivity and specificity of RT-PCR such as DMSO (preferably about 20%), formamide, betaine, trehalose, low molecular weight amides, sulfones or a PCR enhancing factor (PEF). DMSO is preferred.

The composition or kit of the present invention may further comprise a DNA binding protein, such as gene 32 protein from bacteriophage T4 (WO 00/55307, incorporated herein by reference), and the E. coli SSB protein. Other protein additives can include Archaeal PCNA, RNAse H, an exonuclease or another reverse transcriptase. The kit can also comprise an Archaeal DNA polymerase LYP mutant (e.g., L408 mutant of JDF-3 polymerase, L409 mutant of Pfu DNA polymerase) fusion in which the DNA polymerase is fused, for example, to Ncp7, recA, Archacal sequence non-specific double stranded DNA binding proteins (e.g., Sso7d from Sulfolobus solfactaricus, WO 01/92501, incorporated herein by reference), or helix-hairpin-helix domains from topoisomerase V (Pavlov et al., PNAS, 2002).

The composition or kit may also contain one or more of the following items: polynucleotide precursors, primers, buffers, instructions, and controls. Kits may include containers of reagents mixed together in suitable proportions for performing the methods in accordance with the invention. Reagent containers preferably contain reagents in unit quantities that obviate measuring steps when performing the subject methods.

Application in Amplification Reactions

Reverse transcription of an RNA template into cDNA is an integral part of many techniques used in molecular biology. Accordingly, the reverse transcription procedures, compositions, and kits provided in the present invention find a wide variety of uses. For example, it is contemplated that the reverse transcription procedures and compositions of the present invention are utilized to produce cDNA inserts for cloning into cDNA library vectors (e.g., lambda gt10 [Huynh et al., In DNA Cloning Techniques: A Practical Approach, D. Glover, ed., IRL Press, Oxford, 49, 1985], lambda gtl 1 [Young and Davis, Proc. Nat'l. Acad. Sci., 80:1194, 1983], pBR322 [Watson, Gene 70:399-403, 1988], pUC19 [Yarnisch-Perron et al., Gene 33:103-119, 1985], and M13 [Messing et al., Nucl. Acids. Res. 9:309-321, 1981]). The present invention also finds use for identification of target RNAs in a sample via RT-PCR (e.g., U.S. Pat. No. 5,322,770, incorporated herein by reference). Additionally, the present invention finds use in providing cDNA templates for techniques such as differential display PCR (e.g., Liang and Pardee, Science 257(5072):967-71 (1992). The DNA polymerase with increased RT activity, compositions or kits comprising such polymerase can be applied in any suitable applications, including, but not limited to the following examples.

1. Reverse Transcription

The present invention contemplates the use of thermostable DNA polymerase for reverse transcription reactions. Accordingly, in some embodiments of the present invention, thermostable DNA polymerases having increased RT activity are provided. In some embodiments, the thermostable DNA polymerase is selected from the DNA polymerases listed in Tables II-IV, for example, a Pfu or a JDF-3 DNA polymerase.

In some embodiments of the present invention, where a DNA polymerase with increased RT activity is utilized to reverse transcribe RNA, the reverse transcription reaction is conducted at about 50° C. to 80° C., preferably about 60° C. to 75° C. Optimal reaction temperature for each DNA polymerase is know in the art and may be relied upon as the optimal temperature for the mutant DNA polymerases of the present invention. Preferred conditions for reverse transcription are 1X MMLV RT buffer (50 mM Tris pH 8.3, 75 mM KCl, 10 mM DTT, 3 mM MgCl₂), containing 20% DMSO.

In still further embodiments, reverse transcription of an RNA molecule by a DNA polymerase with increased RT activity results in the production of a cDNA molecule that is substantially complementary to the RNA molecule. In other embodiments, the DNA polymerase with increased RT activity then catalyzes the synthesis of a second strand DNA complementary to the cDNA molecule to form a double stranded DNA molecule. In still further embodiments of the present invention, the DNA polymerase with increased RT activity catalyzes the amplification of the double stranded DNA molecule in a PCR as described below. In some embodiments, PCR is conducted in the same reaction mix as the reverse transcriptase reaction (i.e., a single tube reaction is performed). In other embodiments, PCR is performed in a separate reaction mix on an aliquot removed from the reverse transcription reaction (i.e., a two tube reaction is performed).

In another embodiment, the DNA polymerase mutants of the invention can be used for labeling cDNA for microarray analysis, e.g., with fluorescent labels such as Cy3, Cy5 or other labels. It is contemplated that DNA polymerase mutants as described herein would have the advantage of more efficient labeling or more uniform incorporation of labeled nucleotides relative to wild-type enzymes.

2. RT-PCR and PCR

The DNA polymerase with increased RT activity of the present invention is useful for RT-PCR because the reverse transcription reaction may be conducted in a temperature that is compatible with PCR amplification. Another advantage is the possibility of using the same enzyme for cDNA synthesis and PCR amplification. Further, the high temperature at which the thermostable archaeal DNA polymerases function allows complete denaturation of RNA secondary structure, thereby enhancing processivity. The present invention contemplates single-reaction RT-PCR wherein reverse transcription and amplification are performed in a single, continuous procedure. The RT-PCR reactions of the present invention serve as the basis for many techniques, including, but not limited to diagnostic techniques for analyzing mRNA expression, synthesis of cDNA libraries, rapid amplification of cDNA ends (i.e., RACE) and other amplification-based techniques known in the art. Any type of RNA may be reverse transcribed and amplified by the methods and reagents of the present invention, including, but not limited to RNA, rRNA, and mRNA. The RNA may be from any source, including, but not limited to, bacteria, viruses, fungi, protozoa, yeast, plants, animals, blood, tissues, and in vitro synthesized nucleic acids.

The DNA polymerase with increased RT activity of the present invention provides suitable enzymes for use in the PCR. The PCR process is described in U.S. Pat. Nos. 4,683,195 and 4,683,202, the disclosures of which are incorporated herein by reference. In some embodiments, at least one specific nucleic acid sequence contained in a nucleic acid or mixture of nucleic acids is amplified to produce double stranded DNA. Primers, template, nucleoside triphosphates, the appropriate buffer and reaction conditions, and polymerase are used in the PCR process, which involves denaturation of target DNA, hybridization of primers and synthesis of complementary strands. The extension product of each primer becomes a template for the production of the desired nucleic acid sequence. If the polymerase employed in the PCR is a thermostable enzyme, then polymerase need not be added after each denaturation step because heat will not destroy the polymerase activity. Use of thermostable DNA polymerase with increased RT activity allows repetitive heating/cooling cycles without the requirement of fresh enzyme at each cooling step. This represents a major advantage over the use of mesophilic enzymes (e.g., Klenow), as fresh enzyme must be added to each individual reaction tube at every cooling step.

In some embodiments of the present invention, primers for reverse transcription also serve as primers for amplification. In other embodiments, the primer or primers used for reverse transcription are different than the primers used for amplification. In some embodiments, more than one RNA in a mixture of RNAs may be amplified or detected by RT-PCR. In other embodiments, multiple RNAs in a mixture of RNAs may be amplified in a multiplex procedure (e.g., U.S. Pat. No. 5,843,660, incorporated herein by reference).

In addition to the subject enzyme mixture, one of ordinary skill in the art may also employ other PCR parameters to increase the fidelity of synthesis/amplification reaction. It has been reported PCR fidelity may be affected by factors such as changes in dNTP concentration, units of enzyme used per reaction, pH, and the ratio of Mg²⁺ to dNTPs present in the reaction. The fidelity of the reverse transcription step can be increased by adding an exonuclease to the reverse transcription, or the exonuclease activity of polymerase mutants described herein (e.g., L408 mutants of JDF-3 polymerase, L409 mutants of Pfu polymerase) could be used to excise mispaired nucleotides in the DNA/RNA duplex.

Mg²⁺ concentration affects the annealing of the oligonucleotide primers to the template DNA by stabilizing the primer-template interaction, it also stabilizes the replication complex of polymerase with template-primer. It can therefore also increase non-specific annealing and produce undesirable PCR products (giving multiple bands on a gel). When non-specific amplification occurs, Mg²⁺ may need to be lowered or EDTA can be added to chelate Mg²⁺ to increase the accuracy and specificity of the amplification.

Other divalent cations such as Mn²⁺, or Co²⁺ can also affect DNA polymerization. Suitable cations for each DNA polymerase are known in the art (e.g., in DNA Replication 2^{nd} edition, supra). Divalent cation is supplied in the form of a salt such MgCl₂, Mg(OAc)₂, MgSO₄, MnCl₂, Mn(OAc)₂, or MnSO₄. Usable cation concentrations in a Tris-HCl buffer are for MnCl₂ from 0.5 to 7 mM, preferably, between 0.5 and 2 mM, and for MgCl₂ from 0.5 to 10 mM. Usable cation concentrations in a Bicine/KOAc buffer are from 1 to 20 mM for Mn(OAc)₂, preferably between 2 and 5 mM.

Monovalent cation required by DNA polymerase may be supplied by the potassium, sodium, ammonium, or lithium salts of either chloride or acetate. For KCI, the concentration is between 1 and 200 mM, preferably the concentration is between 40 and 100 mM, although the optimum concentration may vary depending on the polymerase used in the reaction.

Deoxyribonucleotide triphosphates (dNTPs) are added as solutions of the salts of dATP, dCTP, dGTP and dTTP, such as disodium or lithium salts. In the present methods, a final concentration in the range of 1 μM to 2 mM each is suitable, and 100-600 μM is preferable, although the optimal concentration of the nucleotides may vary in the PCR reaction depending on the total dNTP and divalent metal ion concentration, and on the buffer, salts, particular primers, and template. For longer products, i.e., greater than 1500 bp, 500 μM each dNTP may be preferred when using a Tris-HCl buffer.

dNTPs chelate divalent cations, therefore amount of divalent cations used may need to be changed according to the dNTP concentration in the reaction. Excessive amount of dNTPs (e.g., larger than 1.5 mM) can increase the error rate and possibly inhibit DNA polymerases. Lowering the dNTP (e.g., to 10-50 μM) may therefore reduce error rate. PCR reaction for amplifying larger size template may need more dNTPs.

One suitable buffering agent is Tris-HCI, preferably pH 8.3, although the pH may be in the range 8.0-8.8. The Tris-HCl concentration is from 5-250 mM, although 10-100 mM is most preferred. Other preferred buffering agents are Bicine-KOH and Tricine.

Denaturation time may be increased if template GC content is high. Higher annealing temperature may be needed for primers with high GC content or longer primers. Gradient PCR is a useful way of determining the annealing temperature. Extension time should be extended for larger PCR product amplifications. However, extension time may need to be reduced whenever possible to limit damage to enzyme.

The number of cycles can be increased if the number of template DNA molecules is very low, and decreased if a higher amount of template DNA is used.

PCR enhancing factors may also be used to improve efficiency of the amplification. As used herein, a “PCR enhancing factor” or a “Polymerase Enhancing Factor” (PEF) refers to a complex or protein possessing polynucleotide polymerase enhancing activity (Hogrefe et al., 1997, Strategies 10::93-96; and U.S. Pat. No. 6,183,997, both of which are incorporated herein by reference). For Pfu DNA polymerase, PEF comprises either P45 in native form (as a complex of P50 and P45) or as a recombinant protein. In the native complex of Pfu P50 and P45, only P45 exhibits PCR enhancing activity. The P50 protein is similar in structure to a bacterial flavoprotein. The P45 protein is similar in structure to dCTP deaminase and dUTPase, but it functions only as a dUTPase converting dUTP to dUMP and pyrophosphate. PEF, according to the present invention, can also be selected from the group consisting of: an isolated or purified naturally occurring polymerase enhancing protein obtained from an archeabacteria source (e.g., Pyrococcus furiosus); a wholly or partially synthetic protein having the same amino acid sequence as Pfu P45, or analogs thereof possessing polymerase enhancing activity; polymerase-enhancing mixtures of one or more of said naturally occurring or wholly or partially synthetic proteins; polymerase-enhancing protein complexes of one or more of said naturally occurring or wholly or partially synthetic proteins; or polymerase-enhancing partially purified cell extracts containing one or more of said naturally occurring proteins (U.S. Pat. No. 6,183,997, supra). The PCR enhancing activity of PEF is defined by means well known in the art. The unit definition for PEF is based on the dUTPase activity of PEF (P45), which is determined by monitoring the production of pyrophosphate (PPi) from dUTP. For example, PEF is incubated with dUTP (10 mM dUTP in 1× cloned Pfu PCR buffer) during which time PEF hydrolyzes dUTP to dUMP and PPi. The amount of PPi formed is quantitated using a coupled enzymatic assay system that is commercially available from Sigma (#P7275). One unit of activity is functionally defined as 4.0 nmole of PPi formed per hour (at 85° C.).

Other PCR additives may also affect the accuracy and specificity of PCR reaction. EDTA less than 0.5 mM may be present in the amplification reaction mix. Detergents such as Tween-20™ and Nonidet™ P-40 are present in the enzyme dilution buffers. A final concentration of non-ionic detergent approximately 0.1% or less is appropriate, however, 0.01-0.05% is preferred and will not interfere with polymerase activity. Similarly, glycerol is often present in enzyme preparations and is generally diluted to a concentration of 1-20% in the reaction mix. Glycerol (5-10%), formamide (1-5%) or DMSO (2-20%) can be added in PCR for template DNA with high GC content or long length (e.g., >1 kb). DMSO, preferably at about 20%, can be added for the cDNA synthesis step using mutant archaeal polymerases described herein. These additives change the Tₘ (melting temperature) of primer-template hybridization reaction and the thermostability of the polymerase enzyme. BSA (up to 0.8 μg/μl) can improve the efficiency of the PCR reaction. Betaine (0.5-2M) is also useful for PCR of long templates or those with a high GC content. Tetramethylammonium chloride (TMAC, >50 mM), Tetraethylammonium chloride (TEAC), and Trimethlamine N-oxide (TMANO) may also be used. Test PCR reactions may be performed to determine optimum concentration of each additive mentioned above.

LYP motif mutants as described herein (e.g., L408 mutants of JDF-3 polymerase, L409 mutants of Pfu polymerase) can be used for cDNA synthesis and for PCR amplification, however, such polymerase mutants can also be used in a mixture or blend with one or more other enzymes used for PCR, e.g., Taq polymerase, Pfu polymerase, etc. for amplification with enhanced fidelity.

The invention provides for additives including, but not limited to antibodies (for hot start PCR) and ssb (higher specificity). The invention also contemplates mutant Archaeal DNA polymerases in combination with Archaeal accessory factors, for example as described in U.S. Pat. No. 6,333,158 (e.g., F7, PFU-RFC and PFU-RFCLS described therein), and WO 01/09347 (e.g., Archaeal PCNA, Archaeal RFC, Archaeal RFC-p55, Archaeal RFC-p38, Archaeal RFA, Archaeal MCM, Archaeal CDC6, Archaeal FEN-1, Archaeal ligase, Archaeal dUTPase, Archaeal helicases 2-8 and Archaeal helicase dna2 described therein), both of which are incorporated herein by reference in their entireties. Further additives include exonucleases such as Pfu G387P to increase fidelity.

Various specific PCR amplification applications are available in the art (for reviews, see for example, Erlich, 1999, Rev Immunogenet., 1:127-34; Prediger 2001, Methods Mol. Biol. 160:49-63; Jurecic et al., 2000, Curr. Opin. Microbiol. 3:316-21; Triglia, 2000, Methods Mol. Biol. 130:79-83; MaClelland et al., 1994, PCR Methods Appl. 4:S66-81; Abramson and Myers, 1993, Current Opinion in Biotechnology 4:41-47; each of which is incorporated herein by references).

The subject invention can be used in RT-PCR or PCR applications, where the PCR applications include, but are not limited to, i) hot-start PCR which reduces non-specific amplification; ii) touch-down PCR which starts at high annealing temperature, then decreases annealing temperature in steps to reduce non-specific PCR product; iii) nested PCR which synthesizes more reliable product using an outer set of primers and an inner set of primers; iv) inverse PCR for amplification of regions flanking a known sequence. In this method, DNA is digested, the desired fragment is circularized by ligation, then PCR using primer complementary to the known sequence extending outwards; v) AP-PCR (arbitrary primed)/RAPD (random amplified polymorphic DNA). These methods create genomic fingerprints from species with little-known target sequences by amplifying using arbitrary oligonucleotides; vi) RT-PCR which uses RNA-directed DNA polymerase (e.g., reverse transcriptase) to synthesize cDNAs which is then used for PCR. This method is extremely sensitive for detecting the expression of a specific sequence in a tissue or cells. It may also be use to quantify mRNA transcripts; vii) RACE (rapid amplification of cDNA ends). This is used where information about DNA/protein sequence is limited. The method amplifies 3′ or 5′ ends of cDNAs generating fragments of cDNA with only one specific primer each (plus one adaptor primer). Overlapping RACE products can then be combined to produce full length CDNA; viii) DD-PCR (differential display PCR) which is used to identify differentially expressed genes in different tissues. First step in DD-PCR involves RT-PCR, then amplification is performed using short, intentionally nonspecific primers; ix) Multiplex-PCR in which two or more unique targets of DNA sequences in the same specimen are amplified simultaneously. One DNA sequence can be use as control to verify the quality of PCR; x) Q/C-PCR (Quantitative comparative) which uses an internal control DNA sequence (but of different size) which compete with the target DNA (competitive PCR) for the same set of primers; xi) Recusive PCR which is used to synthesize genes. Oligonucleotides used in this method are complementary to stretches of a gene (>80 bases), alternately to the sense and to the antisense strands with ends overlapping (˜20 bases); xii) Asymmetric PCR; xiii) In Situ PCR; xiv) Site-directed PCR Mutagenesis.

It should be understood that this invention is not limited to any particular amplification system. As other systems are developed, those systems may benefit by practice of this invention.

### EXAMPLES

### Example 1

Construction of exo− and exo+ JDF-3 and Pfu DNA polymerase mutants that possess reverse transcriptase activity

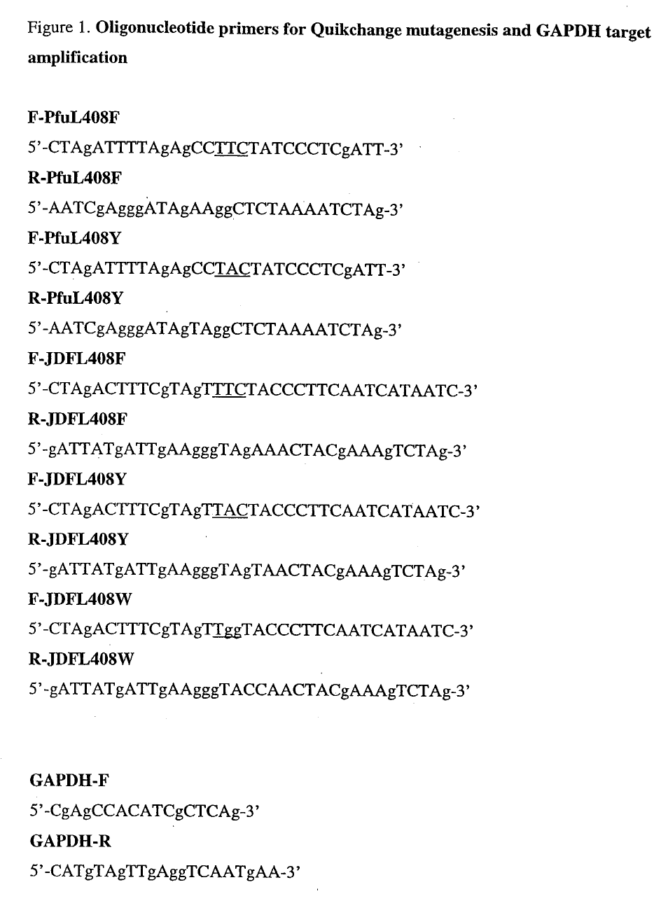

Wild-type (exo⁺) JDF-3 DNA polymerase and JDF-3 DNA polymerase substantially lacking 3′-5′ exonuclease activity (exo⁻) were prepared as described in U.S. patent application Ser. No. 09/896,923. Point mutations phenylalanine (F), tyrosine (Y), and tryptophan (W) were introduced at leucine (L) 409 of exo⁻ and exo⁺ Pfu and at L408 of exo⁻ and exo⁺ JDF-3 DNA polymerases using the Quikchange site directed mutagenesis kit (Stratagene). With the Quikchange kit, point mutations were introduced using a pair of mutagenic primers (FIG. 1). Clones were sequenced to identify the incorporated mutations. Construction of JDF-3 L408H was described previously (see patent application WO 0132887, incorporated herein by reference).

### Example 2

Preparation of bacterial extracts containing mutant JDF-3 and Pfu DNA polymerases

Plasmid DNA was purified with the StrataPrep® Plasmid Miniprep Kit (Stratagene), and used to transform BL26-CodonPlus-RIL cells. Ampicillin resistant colonies were grown up in 1-5 liters of LB media containing Turbo AmPTM (100 μg/μl) and chloramphenicol (30 μg/μl) at 30° C. with moderate aeration. The cells were collected by centrifugation and stored at −80° C. until use.

Cell pellets (12-24 grams) were resuspended in 3 volumes of lysis buffer (buffer A: 50 mM Tris HCl (pH 8.2), 1 mM EDTA, and 10 mM βME). Lysozyme (1 mg/g cells) and PMSF (1 mM) were added and the cells were lysed for 1 hour at 4° C. The cell mixture was sonicated, and the debris removed by centrifugation at 15,000 rpm for 30 minutes (4° C.). Tween 20 and Igepal CA-630 were added to final concentrations of 0.1 % and the supernatant was heated at 72° C. for 10 minutes. Heat denatured E. coli proteins were then removed by centrifugation at 15,000 rpm for 30 minutes (4° C.).

The expression of JDF-3 and Pfu mutants was confirmed by SDS-PAGE (a band migrating at 95 kD).

### Example 3

Evaluation of RT Activity by Radioactive Nucleotide Incorporation Assay

Partially-purified JDF-3 and Pfu mutant preparations (heat-treated bacterial extracts) were assayed to identify the most promising candidates for purification and comprehensive RT-PCR testing. To assess RT activity of the mutants, the relative RNA/DNA dependent DNA polymerization activity was measured for each mutant.

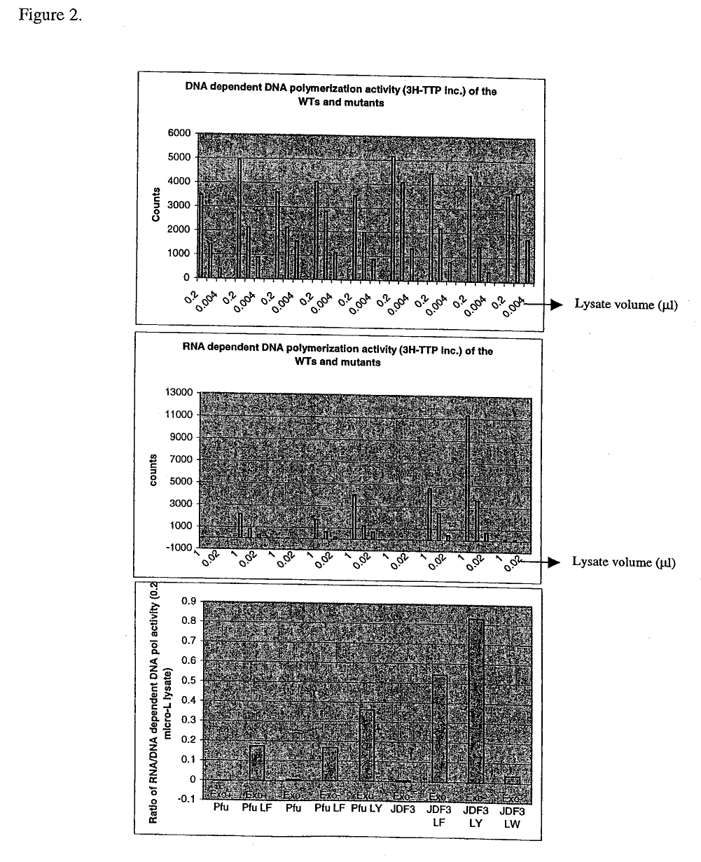

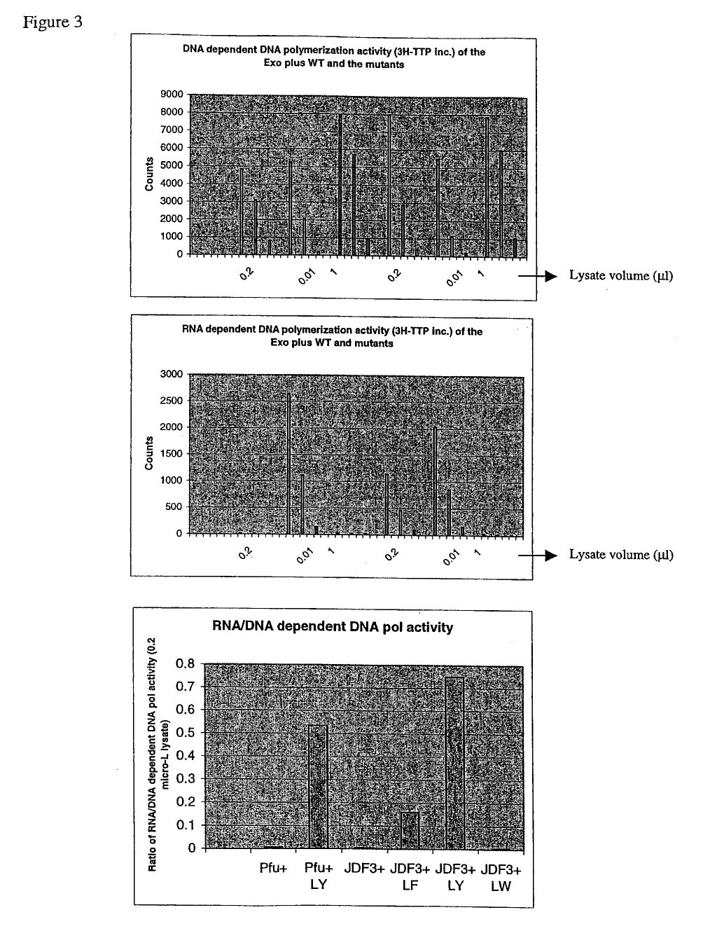

The DNA dependent DNA polymerization activity assay was performed according to a previously published method (Hogrefe, H. H., et al (01) Methods in Enzymology, 343:91-116). Relative dNTP incorporation was determined by measuring polymerase activity ([³H]-TTP incorporation into activated calf thymus DNA). A suitable DNA polymerase reaction cocktail contains: 1× cloned Pfu reaction buffer, 200 μM each dNTPs, 5 μM [³H]TTP (NEN #NET-221H, 1 mCi/ml, 20.5Ci/mmole), 250 μg/ml of activated calf thymus DNA (Pharmacia #27-4575-01. Three different volumes of clarified lysates from WT and mutants (FIGS. 2 and 3) were used in a final reaction volume of 10 μl. Polymerization reactions were conducted in duplicate for 30 minutes at 72° C.

The extension reactions were quenched on ice, and 5 μl aliquots were spotted immediately onto DE81 ion-exchange filters (2.3cm; Whatman #3658323). Unincorporated [³H]TTP was removed by 6 washes with 2×SSC (0.3M NaCl, 30mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms” (wash filters as above). Sample cpms were subtracted by minimum cpms to determine “corrected cpms”.

The RNA dependent DNA polymerization assay was performed as follows. Relative dNTP incorporation was determined by measuring polymerase activity ([³H]-TTP incorporation into poly(dT):poly(rA) template (apbiotech 27-7878)). A suitable DNA polymerase reaction cocktail contains: 1×cloned Pfu reaction buffer, 800 μM TTP, 5 μM [³H]TTP (NEN #NET-601A, 65.8Ci/mmole), 10 μg poly(dT):poly(rA). Three different volumes of clarified lysates from WT and mutants (FIGS. 2 and 3) were used in a final reaction volume of 10 μl. Polymerization reactions were conducted in duplicate for 10 minutes at 50° C. followed by 30 minutes at 72° C.

The extension reactions were quenched on ice, and 5 μl aliquots were spotted immediately onto DE81 ion-exchange filters (2.3 cm; Whatman #3658323). Unincorporated [³H]TTP was removed by 6 washes with 2×SSC (0.3M NaCl, 30 mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms” (wash filters as above). Sample cpms were subtracted by minimum cpms to determine “corrected cpms”.

Partially purified preparations of the exo⁻ and exo⁺ JDF-3 L408F and L408Y and Pfu L409F and L409Y showed improved RT activity compared to wild type JDF-3 and Pfu (FIGS. 2 and 3).

### Example 4

Purification of JDF-3 and Pfu DNA polymerase mutants

JDF-3 and Pfu mutants can be purified as described in U.S. Pat. No. 5,489,523 (purification of the exo⁻ Pfu D141A/E143A DNA polymerase mutant) or as follows. Clarified, heat-treated bacterial extracts were chromatographed on a Q-Sepharose™ Fast Flow column (˜20 ml column), equilibrated in buffer B (buffer A plus 0.1% (v/v) Igepal CA-630, and 0.1% (v/v) Tween 20). Flow-through fractions were collected and then loaded directly onto a P11 Phosphocellulose column (˜20ml), equilibrated in buffer C (same as buffer B, except pH 7.5). The column was washed and then eluted with a 0-0.7M KCI gradient/Buffer C. Fractions containing DNA polymerase mutants (95kD by SDS-PAGE) were dialyzed overnight against buffer D (50 mM Tris HCl (pH 7.5), 5 mM βME, 5% (v/v) glycerol, 0.2% (v/v) Igepal CA-630, 0.2% (v/v) Tween 20, and 0.5M NaCl) and then applied to a Hydroxyapatite column (˜5ml), equilibrated in buffer D. The column was washed and DNA polymerase mutants were eluted with buffer D2 containing 400 mM KPO4, (pH 7.5), 5 mM μME, 5% (v/v) glycerol, 0.2% (v/v) Igepal CA-630, 0.2% (v/v) Tween 20, and 0.5 M NaCl. Purified proteins were spin concentrated using Centricon YM30 devices, and exchanged into final dialysis buffer (50 mM Tris-HCl (pH 8.2), 0.1 mM EDTA, 1 mM dithiothreitol (DTT), 50% (v/v) glycerol, 0.1% (v/v) Igepal CA-630, and 0.1% (v/v) Tween 20).

Protein samples were evaluated for size, purity, and approximate concentration by SDS-PAGE using Tris-Glycine 4-20% acrylamide gradient gels. Gels were stained with silver stain or Sypro Orange (Molecular Probes). Protein concentration was determined relative to a BSA standard (Pierce) using the BCA assay (Pierce).

Mutant proteins were purified to ˜90% purity as determined by SDS-PAGE.

### Example 5

Evaluation of RT Activity of Purified Mutants by Radioactive Nucleotide Incorporation Assay

The RNA dependent DNA polymerization assay was performed as follows. Relative dNTP incorporation was determined by measuring polymerase activity ([³³P]-dGTP incorporation into poly(dG):poly(rC) template (apbiotech 27-7944)). A suitable DNA polymerase reaction cocktail contains: lx cloned Pfu reaction buffer, 800 μM dGTP, 1 μCi [³³ P]dGTP (NEN #NEG-614H, 3000 Ci/mmole), 10 μg poly(dG):poly(rC). The final reaction volume was 10 μl. Polymerization reactions were conducted in duplicate for 10 minutes at 50° C. followed by 30 minutes at 72° C.

The extension reactions were quenched on ice, and 5 μl aliquots were spotted immediately onto DE81 ion-exchange filters (2.3cm; Whatman #3658323). Unincorporated [³³P]dGTP was removed by 6 washes with 2×SSC (0.3M NaCl, 30 mM sodium citrate, pH 7.0), followed by a brief wash with 100% ethanol. Incorporated radioactivity was measured by scintillation counting. Reactions that lacked enzyme were set up along with sample incubations to determine “total cpms” (omit filter wash steps) and “minimum cpms” (wash filters as above). Sample cpms were subtracted by minimum cpms to determine “corrected cpms”.

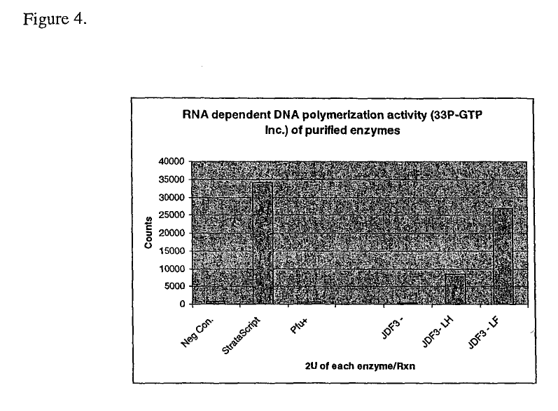

Purified preparations of the exo⁻ JDF-3 L408H and L408F showed improved RT activity compared to wild type JDF-3 and Pfu (FIG. 4). RT activity of 2 units of StrataScript (Stratagene's RNase H minus MMLV-RT) was determined in the same assay for comparison.

### Example 6

Evaluation of RT activity of purified mutants by RT-PCR assay

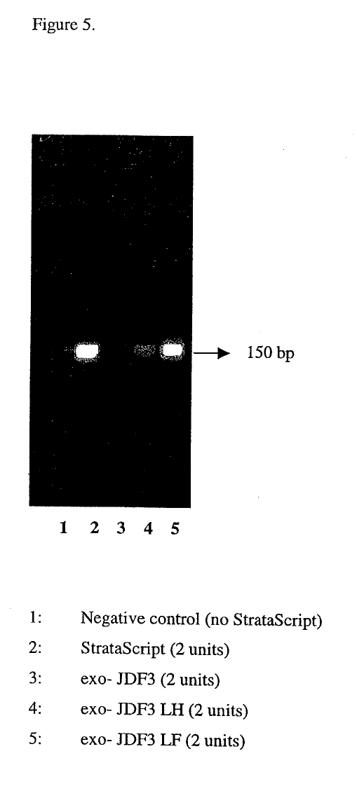

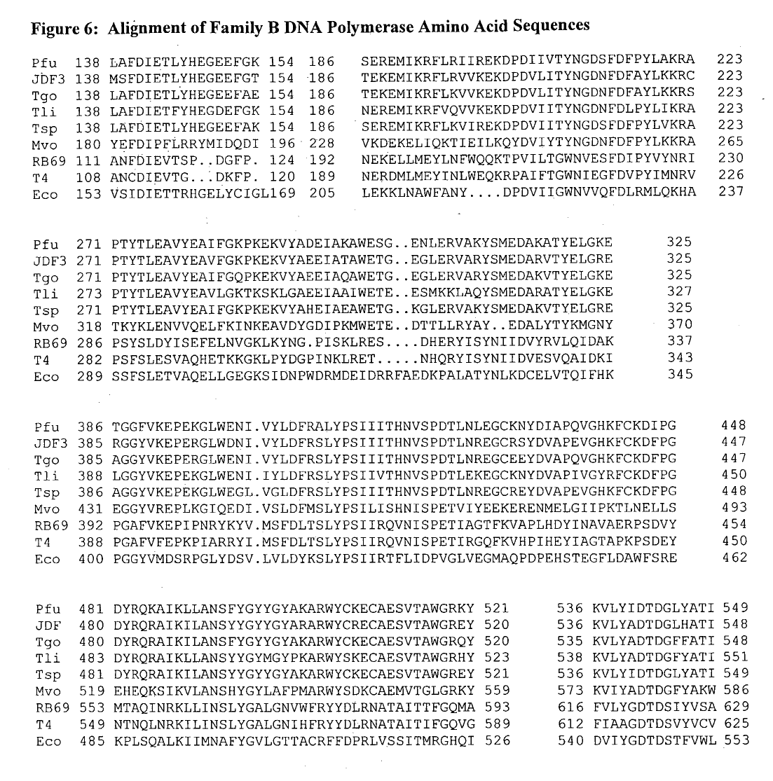

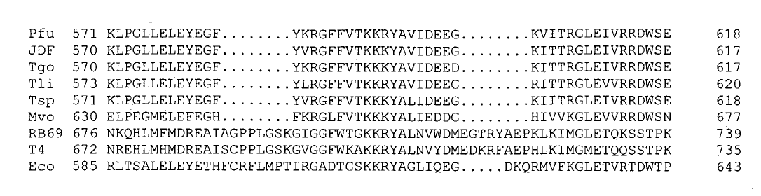

Each RT assay was carried out in a total reaction volume of 10 μl. The final reagent concentrations were as follows: 18 pmol oligo(dT)₁₈, 1 mM each dNTPs, 500 ng human total RNA in either 1× StrataScript buffer (Stratagene) for StrataScript or 1× cloned Pfu buffer (Stratagene) for Pfu, JDF3 WT and mutants. StrataScript reactions were incubated at 42° C. for 40 minutes. WT Pfu, JDF3 and the mutants were incubated at 50° C. for 5 minutes followed by 72° C. for 30 minutes. 2 μl of each cDNA synthesis reaction was used in a PCR containing 2.5 units Taq DNA polymerase, 200 μM each dNTP, 100 ng of each of GAPDH-F and GAPDH-R primers (FIG. 1) in 1×Taq 2000 buffer (Stratagene). Amplification reactions were carried out using the temperature cycling profile as follows: 35 cycles of 95° C. for 30 s, 55° C. for 30 s, and 72° for 1 min. 5 μl of each PCR was run on a 1% agarose gel and stained with ethidium bromide (FIG. 5).

Since the DNA amplification portion of each reaction was performed with the same enzyme (Taq), these results demonstrated that exo- JDF3 L408F exhibit higher reverse transcription efficiency than exo⁻ JDF3 L408H (FIG. 5). The RT activity of the exo⁻ JDF3 is similar to the negative control (no StrataScript).

### Example 7

Evaluation of DMSO effect on RT activity of purified exo+ Pfu L409Y

In order to evaluate the effect of DMSO concentration on RT activity of mutant Archaeal DNA polymerase, a cDNA synthesis reaction was carried out using exo+ Pfu L409Y DNA polymerase in the presence of varying amounts of DMSO. Reactions were carried out in a total volume of 20 μl. The final reagent concentrations were as follows: 1000 ng of exo+ Pfu L409Y, 90 pmol oligo(dT)₁₈, 0.8 mM each dNTPs, 3 μg RNA size marker (Ambion, cat. 7150) in 1×StrataScript buffer (Stratagene). A range of 0-25% DMSO was added to the reactions. Reactions were incubated at 50° C. for 3 minutes followed by 65° C. for 60 minutes. The entire volume of each reaction was run on a 1% alkaline agarose gel and stained with ethidium bromide.

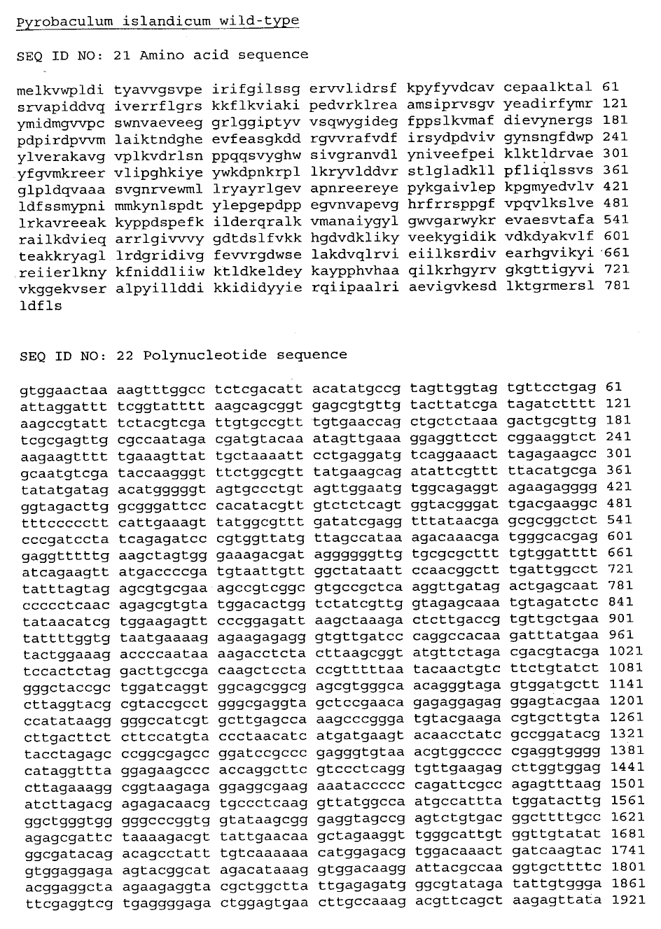

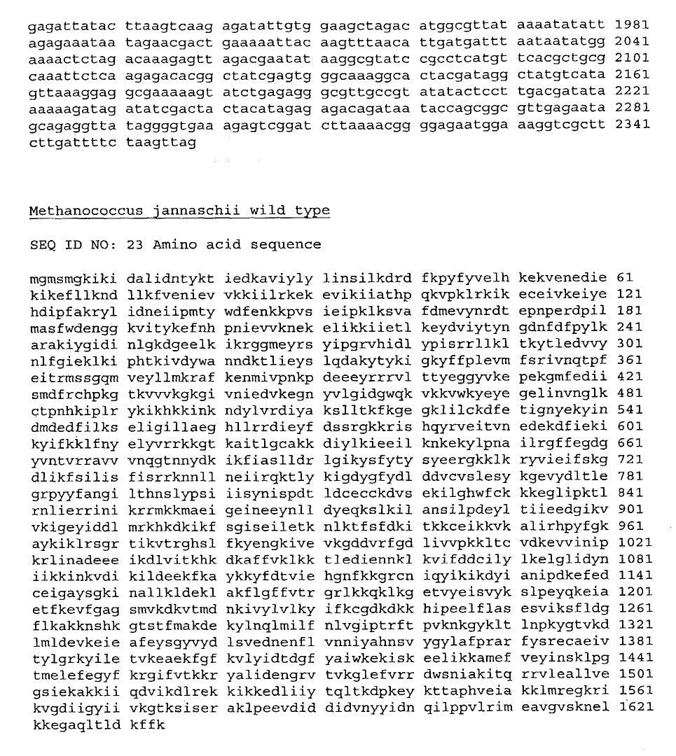

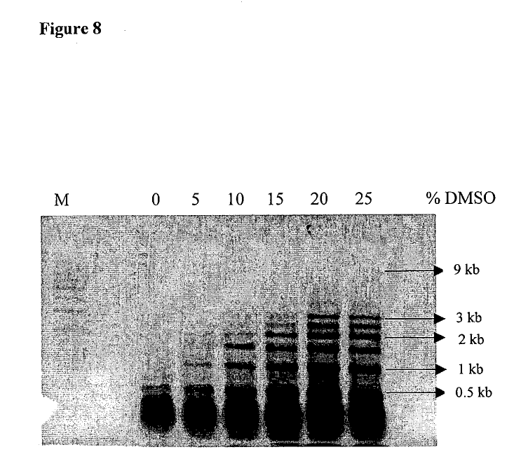

The results shown in FIG. 8 demonstrate that adding DMSO significantly improves the reverse transcriptase activity of exo+ Pfu L409Y.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A recombinant mutant Archacal DNA polymerase exhibiting an increased reverse transcriptase activity.

2. The Archaeal DNA polymerase of claim 1, wherein said DNA polymerase is a mutant of an Archaeal DNA polymerase selected from the group consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9°N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

3. A recombinant mutant Archaeal DNA polymerase exhibiting an increased reverse transcriptase activity, wherein said wild-type form comprises an amino acid sequence selected from SEQ ID Nos. 1, 3, 5, 7, 9, 11, 13, 15, 17, 19, 21 and 23.

4. The Archaeal DNA polymerase of claim 1 or 3, comprising an amino acid mutation at the amino acid corresponding to L408 of SEQ ID NO: 1.

5. The Archaeal DNA polymerase of claim 4, wherein said amino acid mutation at the poisition corresponding to L408 of SEQ ID NO: 1 is a leucine to phenylalanine mutation, leucine to tyrosine mutation, leucine to histidine mutation or a leucine to tryptophan mutation.

6. The mutant Archaeal DNA polymerase of claim 1 or 3, further exhibiting a decreased 3′-5′ exonuclease activity.

7. The mutant Archaeal DNA polymerase of claim 1 or 3, further exhibiting a reduction in non-conventional nucleotide discrimination.

8. A chimeric polypeptide comprising a mutant Archaeal DNA polymerase and a second polypeptide fused to said mutant Archaeal DNA polymerase, wherein said mutant Archaeal DNA polymerase exhibits an increased reverse transcriptase activity.

9. The chimeric polypeptide of claim 8, wherein said second polypeptide is fused to the N- or C-terminus of said mutant Archaeal DNA polymerase.

10. The chimeric polypeptide of claim 8, wherein said second polypeptide is a polynucleotide binding protein.

11. The chimeric polypeptide of claim 10, wherein said polynucleotide binding protein is selected from the group consisting of: nucleocapsid protein Ncp7, recA, SSB, T4 gene 32 protein, an Archaeal non-sequence specific double stranded DNA binding protein, and a helix-hairpin-helix domain.

12. The chimeric polypeptide of claim 11, wherein said Archaeal sequence non-specific double stranded DNA binding protein is selected from Sso7d, Sac7d and PCNA.

13. The chimeric polypeptide of claim 11, wherein said helix-hairpin-helix domain is from topoisomerase V.

14. An isolated polynucleotide encoding a mutant Archaeal DNA polymerase which exhibits an increased reverse transcriptase activity.

15. The isolated polynucleotide of claim 14, wherein said Archaeal DNA polymerase is selected from the group consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

16. An isolated polynucleotide encoding a mutant Archaeal DNA polymerase which exhibits an increased reverse transcriptase activity compared to a DNA polymerase encoded by a wild-type polynucleotide, wherein said wild-type polynucleotide comprises a sequence selected from the group consisting of SEQ ID Nos. 2, 4, 6, 8, 10, 12, 14, 16, 18, 20 and 22.

17. The polynucleotide of claim 14 or 16, wherein said Archaeal DNA polymerase comprises an amino acid mutation at the amino acid corresponding to L408 of SEQ ID NO: 1.

18. The polynucleotide of claim 17, wherein said amino acid mutation at the amino acid corresponding to L408 of SEQ ID NO: 1 is a leucine to phenylalanine mutation, leucine to tyrosine mutation, leucine to histidine mutation or a leucine to tryptophan mutation.

19. An isolated polynucleotide encoding a chimeric polypeptide of either of claims 8 or 14.

20. A composition comprising a mutant Archaeal DNA polymerase exhibiting an increased reverse transcriptase activity.

21. The composition of claim 20, wherein said Archaeal DNA polymerase is selected from the group consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9° N-7 DNA polymerase; Pyrodictium occultum DNA polymerase; Methanococcus voltac DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

22. A composition comprising a mutant Archaeal DNA polymerase exhibiting an increased reverse transcriptase activity, wherein the wild-type form of that Archaeal DNA polymerase comprises an amino acid sequence selected from SEQ ID Nos. 1, 3, 5, 7, 9, 11, 13, 15, 17, 19, 21 and 23.

23. The composition of claim 20 or 22, wherein said Archaeal DNA polymerase comprises an amino acid mutation at the amino acid corresponding to L408 of SEQ ID NO: 1.

24. The composition of claim 23, wherein said amino acid mutation at the amino acid corresponding to L408 of SEQ ID NO: 1 is a leucine to phenylalanine mutation, a leucine to tyrosine mutation, a leucine to histidine mutation, or a leucine to tryptophan mutation.

25. The composition of claim 20 or 22, further comprising one or more reagents selected from the group consisting of: reaction buffer, dNTP, control RNA template and control primers.

26. The composition of claim 20 or 22, further comprising one or more reagents selected from the group consisting of: formamide, DMSO, betaine, trehalose, low molecular weight amides, sulfones, an Archaeal accessory factor, a single-stranded DNA binding protein, a DNA polymerase, another reverse transcriptase enzyme, and an exonuclease.

27. A kit comprising a mutant Archaeal DNA polymerase exhibiting an increased reverse transcriptase activity, and packaging materials therefor.

28. The kit of claim 27, wherein said Archaeal DNA polymerase is selected from the group consisting of: Thermococcus litoralis DNA polymerase (Vent); Pyrococcus sp. DNA polymerase (Deep Vent); Pyrococcus furiosus DNA polymerase (Pfu); JDF-3 DNA polymerase; Sulfolobus solfataricus DNA polymerase (Sso); Thermococcus gorgonarius DNA polymerase (Tgo); Thermococcus species TY DNA polymerase; Thermococcus species strain KODI (KOD) DNA polymerase; Thermococcus acidophilium DNA polymerase; Sulfolobus acidocaldarius DNA polymerase; Thermococcus species 9° N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; and Aeropyrum pernix DNA polymerase.

29. A kit comprising a mutant Archaeal DNA polymerase exhibiting an increased reverse transcriptase activity, wherein the wild-type form of that Archaeal DNA polymerase comprises an amino acid sequence selected from SEQ ID Nos. 1, 3, 5, 7, 9, 11, 13, 15, 17, 19, 21 and 23.

30. The kit of claim 27 or 29, wherein said Archaeal DNA polymerase comprises an amino acid mutation at the amino acid corresponding to L408 of SEQ ID NO: 1.

31. The kit of claim 30, wherein said amino acid mutation at the amino acid corresponding to L408 of SEQ ID NO: 1 is a leucine to phenylalanine mutation, leucine to tyrosine mutation, leucine to histidine mutation or a leucine to tryptophan mutation.

32. The kit of claim 27 or 29, further comprising one or more reagents selected from the group consisting of: reaction buffer, dNTP, control RNA template and a control primer.

33. The kit of claim 27 or 29, further comprising one or more reagent selected from the group consisting of: formamide, DMSO, betaine, trehalose, low molecular weight amides, sulfones, an Archaeal accessory factor, a single-stranded DNA binding protein, a DNA polymerase other than said mutant Archaeal DNA polymerase, another reverse transcriptase enzyme, and an exonuclease.

34. A method for reverse transcribing an RNA template, comprising incubating said RNA template in a reaction mixture comprising a mutant Archaeal DNA polymerase exhibiting an increased reverse transcriptase activity, wherein said incubation permits reverse transcription of said RNA template.

35. A method for amplifying an RNA, comprising incubating said RNA template in a reaction mixture comprising a mutant Archaeal DNA polymerase exhibiting an increased reverse transcriptase activity, wherein said incubation permits amplification of said RNA template.

36. A method for amplifying an RNA, comprising: (a) incubating said RNA template in a first reaction mixture comprising a mutant Archaeal DNA polymerase exhibiting an increased reverse transcriptase activity, wherein said incubation permits reverse transcription of said RNA template to generate a cDNA template; and (b) incubating said cDNA template in a second reaction mixture, wherein that incubating permits amplification of said cDNA template.

37. The method of claim 36 wherein said second reaction mixture further comprises a second DNA polymerase or a combination of two or more other DNA polymerases.

38. The method of claim 37 wherein said second DNA polymerase is a wild-type DNA polymerase.

39. The method of claim 37 wherein said second DNA polymerase comprises Taq DNA polymerase, Pfu Turbo DNA polymerase or a combination of these two.
